(19) United States
(12) Patent Application Publication
(10) Pub. No.: US 2013/0312897 A1
(43) Pub. Date: Nov. 28, 2013

(54) PROSTHETIC MENISCI AND METHOD OF IMPLANTING IN THE HUMAN KNEE JOINT

(71) Applicant: Robert Walter Vowles, Romsey (AU)

(72) Inventor: Robert Walter Vowles, Romsey (AU)

(73) Assignee: PROSTHEXIS PTY LTD, Romsey, Victoria (AU)

(21) Appl. No.: 13/984,267

(22) PCT Filed: Feb. 8, 2012
(86) PCT No.: PCT/AU12/00119

§ 371 Date: Aug. 7, 2013

(30) Foreign Application Priority Data
Feb. 8, 2011 (AU) 2011900401

(51) Int. Cl. A61F 2/30; A61B 8/08; A61B 6/00; A61B 5/055

(52) U.S. Cl. CPC A61F 2/30756; A61F 2/3094; A61B 5/055; A61B 8/08; A61B 6/50
USPC 156/83; 156/245; 623/14.12

(57) ABSTRACT

A method of making prosthetic knee menisci to be implanted in place of deteriorated or damaged native menisci, said prosthetic menisci being internally reinforced and sequentially moulded in open form from a suitable biocompatible elastomeric polymer material, said prosthetic menisci subsequently being transformed into closed, hollow forms; said hollow forms being sized for femoral and tibial condylar surfaces and filled or inflated after implantation by injection into them of a gel or settable polymer to shape them into congruence with the femoral and tibial condyles, the surfaces of said menisci being chemically and/or physically treated to improve the efficiency of lubrication by synovial fluid and to provide enhanced wear characteristics, and said prosthetic menisci being restricted in translation within the interarticular space by anchorage of their anterior and posterior horns to the tibia and by the provision of secondary locating elements.

This invention relates generally to surgical methods of alleviating the discomfort and impaired mobility resulting from deterioration or injury to the meniscus of the human knee. More specifically, it relates to a method of replacing either or both of the native menisci in the human knee with prosthetic menisci.

Arthritic deterioration of the knee joint is now extremely common in the western world. A progressively aging population is doubtless a contributory factor. As a result of the limited options available, the large number of total knee joint replacements being performed annually in many countries is becoming a very substantial burden on the healthcare system. Worldwide, the number of procedures is increasing almost exponentially, despite the fact that it is irreversible and may ultimately require revision. Indeed, the number of total knee replacement revisions has become so great that it now constitutes a well defined sub-speciality in orthopaedic surgery. Clearly, any alternatives to forestall the need for total knee replacement should be considered.

The knee complex is one of the most frequently injured joints in the human body. The knee joint works in conjunction with the hip joint and ankle to support the weight of the body during static, erect posture. Dynamically, it is responsible for moving and supporting the body during a variety of both routine and difficult activities. The fact that the knee must fulfil both major stability and major mobility functions is reflected in its complex structure and functionality.

The two major bones of the leg are the femur, the proximal end of which pivots at the hip joint and the tibia, the distal end of which pivots at the ankle joint. The femur and tibia pivot are joined in an articulated relationship at the knee by the tibiofemoral joint, the largest in the body. The distal end of the femur and proximal end of the tibia are expanded and, although this provides some basis for stability, there is no great adaptation of the bony ends one to another. The distal end of the femur is developed into two discrete condyles, the lower surfaces of which are smoothly rounded and covered in (hyaline) articular cartilage which provides a smooth bearing surface. The anteroposterior convexity of the condyles is not consistently spherical, having a smaller radius of curvature posteriorly.

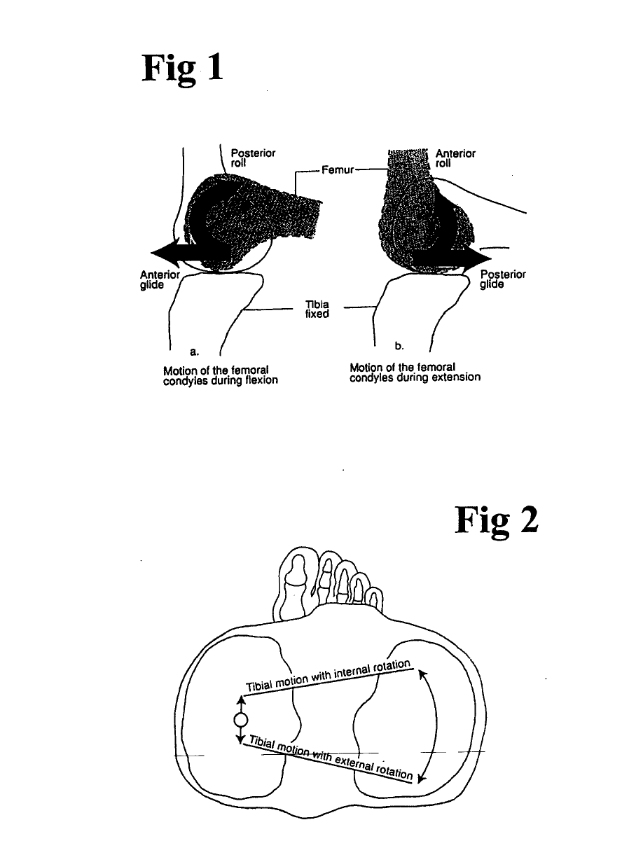

The cruciate ligaments of the knee act to substantially locate the condyles on the tibia during flexion and extension. As illustrated in a. of FIG. 1, during flexion of the knee joint, tension applied by the anterior cruciate ligament restrains the condyles from posterior displacement. Similarly, as illustrated in b. of FIG. 1, during extension of the knee joint, the posterior cruciate ligament restrains the condyles from anterior displacement. These effects are reinforced by the capsule and the layers of ligamentous and tendinous tissue surrounding the knee joint.

Medial and lateral rotation of the knee joint are angular motions that are named for the motion (or relative motion) of the tibia on the femur. These axial rotations of the knee joint occur about a longitudinal axis that runs through or close to the medial tibial intercondylar tubercle. Consequently, the medial condyles acts as pivot points while the lateral condyles move through a greater arc of motion, regardless of the direction of rotation. This is illustrated in FIG. 2. As the tibia laterally rotates on the femur, the medial tibial condyle moves only slightly anteriorly on the relatively fixed medial femoral condyle, whereas the lateral tibial condyle moves a larger distance posteriorly on the relatively fixed lateral femoral condyle. During tibial medial rotation, the medial tibial condyle moves only slightly posteriorly, whereas the lateral condyle moves anteriorly through a longer arc of motion. During both medial and lateral rotation, the knee joint menisci will distort in the direction of movement of the corresponding femoral condyle and, therefore, maintain their relationship to the femoral condyles as they did in flexion and extension.

Meniscal motion on the tibia is limited by multiple attachments to surrounding structures, some common to both menisci and some unique to each. To accommodate deviations of the femoral condyles from sphericality, the menisci obviously possess some freedom of movement. The medial meniscus has greater ligamentous and capsular constraints, limiting its translation to a greater extent than that of the lateral meniscus. The relative lack of mobility of the medial meniscus may contribute to its greater incidence of injury—some nine times greater than that of the lateral meniscus.

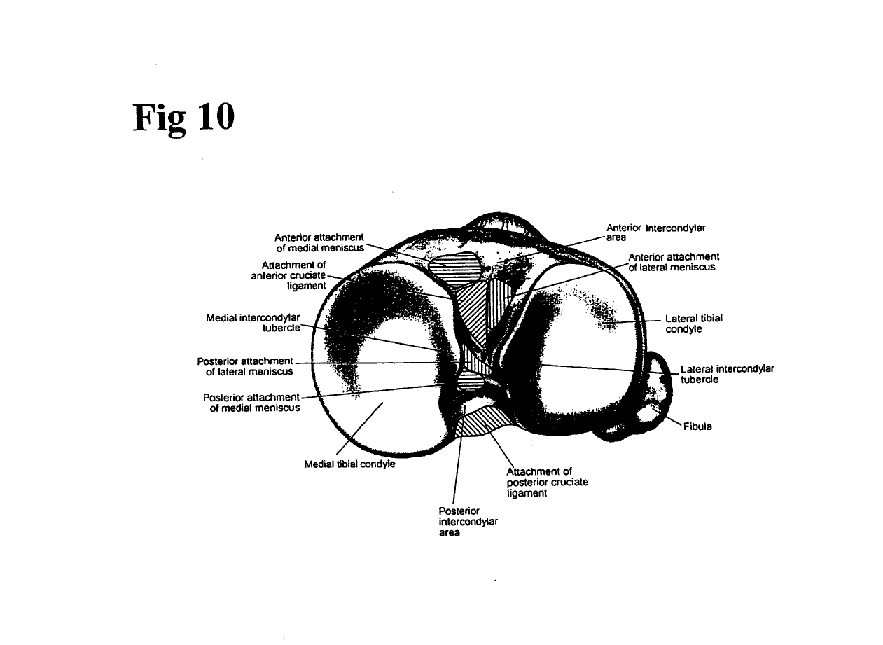

The menisci are best described as crescent-shaped wedges of fibrocartilage supported upon the peripheral aspects of the articular surfaces of the proximal tibia. They function to effectively deepen the medial and lateral tibial fossae for articulation with the condyles of the femur. They are thickest at their external margins and taper to thin, unattached edges as they extend radially inwards. The superior surfaces of the menisci are slightly concave to accommodate the condyles of the femur and providing greater contact surface area. The medial meniscus is larger than the lateral and more ovoid in shape. Anteriorly, it is thin and pointed at its attachment in the anterior intercondylar area of the tibia, directly outside the anterior cruciate ligament. Posteriorly, it is broadest, attaching in the corresponding posterior fossa, anteriorly to the origin of the posterior cruciate ligament. The lateral meniscus is smaller and more circular, its anterior horn being attached in the anterior intercondylar area, posteriorly and laterally to the insertion of the anterior cruciate ligament. Its posterior horn terminates in the posterior intercondylar area, immediately anterior to the termination of the posterior horn of the medial meniscus. The lateral meniscus is weakly attached around the margin of the lateral tibial condyle, except where crossed by the popliteal tendon and is not attached to the fibular collateral ligament. The lateral meniscus is thus loosely attached to the tibia and has frequent attachment to the femur. Therefore, it tends to move forward and backward with the lateral femoral condyle during flexion of the knee. In contrast, the medial meniscus is more firmly fixed to the tibia. The convex anterior margin of the lateral meniscus is connected to the anterior horn of the medial meniscus (or its convex anterior margin) by the transverse genicular ligament. This connection allows the two menisci to move in unison. The capsular and tibial attachments of the meniscus may be seen clearly in FIG. 9. The tibial plateaux and meniscal horn attachment sites are illustrated in FIG. 10.

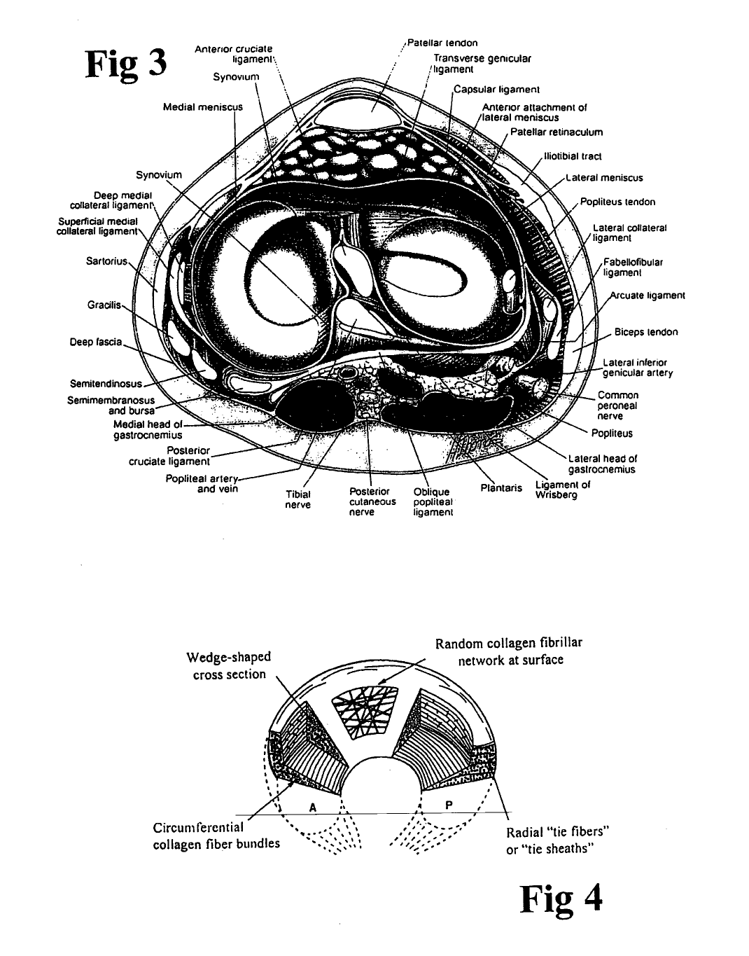

The thick peripheral margins of the menisci have an extensive microvascular network that arises from their respective superior and inferior genicular branches of the popliteal artery, while the thin, unattached edges of the menisci within the joint are avascular. The perimeniscal capillary plexus is oriented circumferentially and it branches extensively into smaller vessels to supply the menisci. The capillaries are developed into smaller vessels which extend peripherally throughout 10 to 30 percent of the medial meniscus and 10 to 25 percent of the lateral meniscus. Similarly, nerve fibres originate in the perimeniscal tissues and radiate into the peripheral 30 percent of the menisci. The most densely innervated regions are the anterior and posterior horns, these nerves being thought to play a proprioceptive role for protective neuromuscular reflex control of joint motion and loading. The location and morphology of the menisci and associated structures are illustrated in FIG. 3.

The anterior glide of the femoral condyles during flexion is also influenced by the menisci. The effective ‘wedging’ effect of the menisci acts to restrain the condyles from posterior displacement while the reaction forces applied to them act to displace the menisci posteriorly on the tibial plateaus. Deformation of the menisci occurs because the rigid attachment of their horns limits their ability to move in their entirety. Posterior deformation permits the menisci to remain beneath the femoral condyles as the condyles move on the tibial plateaus. As the knee returns to extension from full flexion, the menisci return to their neutral positions and, as extension continues, are deformed anteriorly.

The menisci are, effectively, cartilaginous extensions of the tibia composed principally of type I collagen. Water accounts for more than 70 percent of the total weight of the meniscus. FIG. 4 shows the physical structure of the meniscal collagen networks which can be roughly divided into three separate zones. In the outer, superficial layer, fibrils are randomly oriented and are interwoven to form a fine mesh. Immediately beneath this mesh is a narrow zone in which the collagen bundles show a much more irregular orientation. Interior to these two surface zones, the collagen fibres form large bundles that can be seen with the naked eye. These fibre bundles are circumferentially arranged, extending from the anterior attachment site to the posterior attachment site. Between these large, circumferentially arranged collagen fibre bundles are smaller tie fibres or tie sheaths orientated radially and extending from the periphery to the inner edge. Thus, compressive force applied to the meniscus is translated into a circumferentially directed tensile or hoop stress, supported by the strong circumferential fibres that dominate its ultrastructure. Viscoelastic behaviour of meniscus material to tensile and compressive forces is complex, the tensile modulus, stiffness and failure stress correlating with collagen content and ratio of collagen to proteoglycan (PG). When meniscus material is loaded in compression, a loss of volume can occur due to fluid exudation from the tissue and/or fluid distribution within the tissue. The concentration of PG within the tissue has been shown to affect permeability, suggesting a direct relationship between PG content and compressive stiffness. The concentration and molecular conformation of proteoglycan aggregates in cartilage vary with age and disease and the amount of PG present depends on joint loading and motion. In general, with aging and disease, the size of the PG aggregates decreases by shortening of the hyaluronic acid chain or by shortening of either the protein core or glycosaminglycan chains, or both. The net result is a reduction in resilience of the cartilage and a concomitant disposition towards mechanical damage.

The lubrication process of the knee joint is thought to be a combination of boundary lubrication and fluid film lubrication, but modified by the characteristics of the articulating cartilage surfaces. Boundary lubrication depends upon the chemical adsorption of a monolayer of lubricant molecules to the articulating surfaces, the clearance between the articulating surfaces maintained by the lubrication mechanism being as small as only a few μM. During relative motion, the surfaces are protected by the lubricant molecules sliding over one another, preventing adhesion and abrasion of the naturally occurring surface asperities. In fluid film lubrication, a much thicker (10-20 μM) layer of lubricant is necessary, compared with the molecular size of the lubricating glycoprotein molecule. The lubricant layer causes relatively wide separation of the articulating surfaces compared with the typical surface roughness of normal articular cartilage. The load applied across the surfaces is supported by pressure generated in the fluid film. The low relative speed difference of the articulating surfaces and the high loads applied across them are, generally speaking, incompatible with the concept of fluid film lubrication. This has led to the postulation of a ‘weeping lubrication’ process in which lubricant exudes from the permeable cartilage surface as a result of applied pressure. Experimental investigations have been unable to validate this hypothesis and a theory of ‘boosted lubrication’ is now accepted. In this process, high pressures generated in the fluid lubricant film causes synovial fluid without hyaluronate to flow into the cartilage tissue, leaving a concentrated gel in the gap to protect the articulating surfaces. It is also now accepted that micro-elastohydrodynamic lubrication contributes substantially to formation of effective lubricating films in synovial joints. Micro-elastohydrodynamic lubrication occurs when elastomeric layers deform under pressure, forming a fluid film in which asperities in the articulating surfaces are flattened as a result of local pressure perturbations. In light of the ability of synovial joints to maintain a high level of lubrication efficiency under a wide range of conditions, it is not unreasonable to infer the presence of a hybrid lubrication mechanism and it is notable that substantial differences of agreement still exist in relation to the subject. Regardless of the lubricating mechanism, by engineering standards, friction in the diarthrodial joint is reduced to levels associated with a fluid film separating the sliding surfaces (hydrodynamic lubrication), but at sliding velocities normally associated only with boundary (solid-to-solid) lubrication and, hence, with frictional levels one to two orders of magnitude higher. As an indicator of the efficiency of the lubrication system, coefficients of kinetic friction (g) in human joints are approximately 0.002 to 0.006, compared with a value of 0.04 for Teflon, which is one of the best boundary lubricants used in non-biological systems. The coefficient of friction (μ) is the ratio of the frictional force (T) resisting movement of one articulating surface over another and the normal force (N) urging the articulating surfaces together (μ=T/N).

Examination of the knee joint using magnetic resonance imaging has shown the relatively large excursions experienced by the menisci during various phases of knee joint flexion. FIGS. 6, 7 and 8 give some figures in this regard. Given the relatively high degree of meniscal mobility, it will be appreciated that the primary locational mechanism of the femoral condyles on the tibial plateaux is tension applied by the anterior and posterior cruciate ligaments. The menisci essentially provide a moveable, cushioned bearing surface for the femoral condyles and may, at extremes of knee flexion, provide supplementary locational assistance. This factor makes possible the provision of prosthetic menisci which, while not able to accommodate the rigors of athletic performance, will readily meet the needs of a sedentary person of middle age.

Meniscus failure commonly takes two forms: direct mechanical damage and that resulting from degenerative breakdown. In sports persons, for example, acute tearing of the meniscus may result when the knee is bent and forcefully twisted. Degenerative tears in the meniscus are very common in older persons with some 60 percent of western populations over the age of 65 years having some sort of degenerative breakdown. While acute tearing may result in the sudden onset of symptoms, in older subjects, degenerative breakdown may result from minor events and be symptomless for an extended period. A combination of circumstances, such as age-related degenerative changes, ‘wear and tear’ arthritis of the whole knee typically found in former athletes, inflammatory arthritis, decline of synovial lubrication, degradation caused by enzymes, unnatural gait, alignment disorders of the leg or excessive knee loadings as a result of occupational activities may result in progressive frictional wear of a meniscus, the cartilage having very little power of natural restoration. The meniscus is capable of self healing only in the vascularised, innervated peripheral zone while the unattached central zone is nourished only by synovial fluid and, generally speaking, is incapable of self healing.

In light of the foregoing generally, it is postulated that, if arthritic deterioration can be detected at an early enough stage, intervention in the form of the implantation of prosthetic menisci may be sufficient to almost immediately restore normal knee function. Further, this has the potential to arrest the deterioration process and to forestall the eventual need for total or partial knee replacement. If the implantation procedure can be performed arthroscopically as a day procedure, the reduction in demand for hospital bed space and orthopaedic, anaesthetic, physiotherapy and general medical services will represent a significant cost saving. Concomitant benefits would be rapid patient recovery with minimal discomfort and, if required, ease of revision.

The concept of prosthetic menisci is well known. While intended primarily as a scaffold for the regrowth of native meniscal tissue, the prosthetic meniscus taught by Stone in U.S. Pat. No. 4,880,429, U.S. Pat. No. 5,007,934, U.S. Pat. No. 5,116,374 and U.S. Pat. No. 5,158,574 are indicative of the concept. In this example, the prosthetic meniscus is made crescent-shaped or fully circular, wedge-shaped in cross-section and from any biocompatible, bioresorbable fibres. The fibres are optionally ordered or unordered, arranged in varying densities and are, at least partially, cross-linked to preserve the temporary stability and shape of the structure in vivo and to control the rate of resorption. An adhesive portion and glycosaminoglycan molecules are dispersed throughout the fibres, the latter optionally acting to provide intermolecular cross-links, the hydrophilicity and elasticity of the cross-linked meniscus simulating the properties of the natural meniscus. A mesh member extending outwardly from the peripheral edge of the meniscus provides a ready means of anchoring. The material from which the prosthetic meniscus in made is not suitable for the permanent, biostable meniscus. While not stated in the patents, it seems unlikely that a human knee implanted with the prosthetic meniscus could sustain any degree of weight-bearing activity until regeneration and resorption were well advanced, stated to be six weeks in the animal studies referred to. No description is given as to how the necessary high level of lubrication efficiency of the natural and prosthetic cartilage might be achieved prior to cartilage regeneration. Success of the method appears to rely upon retention of a good part of the native meniscus as a foundation for the regeneration.

Another example is that taught by Swartz in U.S. Pat. No. 5,344,459. In this example, a prosthetic device or pair of joined or discrete devices is provided, the devices being principally circular, single or multi-compartmented and inflatable. The devices are optionally fully or partially inflated with air, a non-reactive gas, water, other inert liquid, or a settable solid or semi-solid to provide the desired level of stiffness. Alternatively, the devices take the form of annularly shaped rings which are solid, semi-solid or spongy, without the need for inflation, filling or expansion with a substance, the filling material optionally having layers or regions of differing levels of stiffness or compressibility. Fibre reinforcements are optionally arranged radially around the upper and lower surfaces of the devices. The annular shape of the devices is deemed appropriate for use within the knee joint and it is doubtful that the devices would lend themselves to being re-shaped to properly adapt them. Although the patent states that the devices are implanted arthroscopically, no description is provided as to how this technique might be applied. No provision is made to anchor or control the motion of the devices and it is doubtful that they could function properly without appropriate translational restraint. While the devices optionally incorporate a plurality of discrete, separately inflatable, internal compartments (or compartments which are solid, semi-solid or spongy), no description is given as to how these compartments are located within a device or what fills the space between the compartments and the clearly larger jacket of tough, outer, polymeric material. No description is given as to how the necessary high level of lubrication efficiency is achieved. No reinforcement provision is made to accommodate hoop stresses generated in the devices.

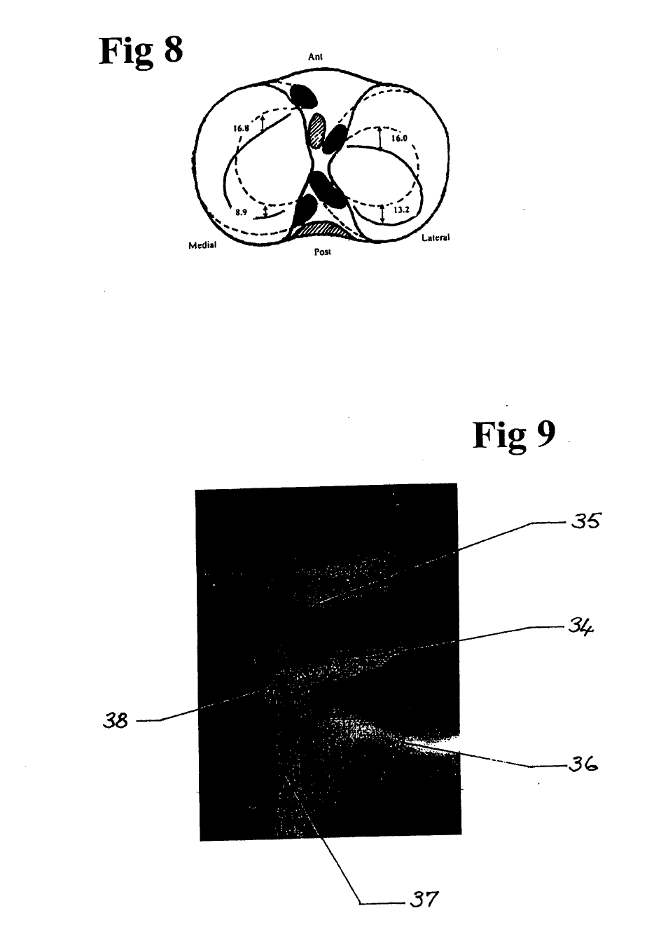

A further example is that taught by Wall in U.S. Pat. No. 4,502,161. In this example, a prosthetic meniscus is provided to replace natural menisci in different types of human joint. The meniscus is optionally made from one of several materials, including silicone rubber, Teflon®, Silastic material, natural rubber and silicone rubber surgical foams. The meniscus is supported in place in a joint by the fixing of an extra-articular extension or strut to an adjacent bone surface. In the case of the knee, the extra-articular extension or strut of a lateral prosthetic meniscus is nominated as being fixed to the femur and that of a medial meniscus as being fixed to the tibia. Reinforcing material, preferably in the form of closely-woven fine fabric mesh of stainless steel, Nylon®, Dacron® or rayon strands or a sheet of perforated material, is embedded in the prosthetic meniscus and carried out into the extra-articular extension or strut. No description is given as to how the necessary high level of lubrication efficiency of the prosthetic meniscus is achieved. The arrangement of the reinforcing material (depicted in FIG. 8 as dividing at the extra-articular extension or strut) would not efficiently accommodate hoop stresses generated in a prosthetic meniscus. Attachment of the extra-articular extension or strut to the femur would seriously inhibit joint movement.

A further example is that taught by Kenny in U.S. Pat. No. 4,344,193, U.S. Pat. No. 5,092,894 and U.S. Pat. No. 5,171,322. The first cited patent teaches a meniscus prosthetic device made with a wedge-shaped cross-sectional shape from an elastic, self-supporting material, such as silicone rubber. The device has on each of its terminal ends a prominence noticeably raised above the general surface of the device. The prominences, when properly placed, superiorly, in the knee, enter the space between the condyles and thus secure the device in position. The nature of the material of the material from which the device is made permits the device to be installed by elastically deforming it, by the application of opposing forces to the terminal ends. This spreading or deformation permits the device to be positioned between the femoral and tibial articular cartilage, whereupon the opposing forces are released and the device allowed to elastically contract with its prominences suitably positioned. The device is optionally made without the prominences and sutures are provided around its periphery to hold the device in the proper position by attachment to adjacent bone or tissue. The device is optionally secured in place with a combination of prominences, sutures and ingrowth of fibrous tissue into a porous border. The second and third cited patents teach a meniscus prosthetic device of wedge-shaped cross-sectional shape made in the form of an arcuate body from a biocompatible, deformable and resilient material. The material is optionally silicone rubber or comparable material, a natural substance, such as animal tissue (collagen, tendon or fibrocartilage), or tissue harvested from the patient's own body, such as periosteal tissue turned inside out or part of a tendon. The arcuate extent of the body of any meniscus prosthetic device is substantially less than 360°, although at least 150° and preferably at least 180°. The device is made with tail means which are optionally discrete and attached to each end of the device; joining both ends of the device; or formed on one end of the device and joinable to the other. The tail means may be of the same material as the body part or a different material and need not be as flexible and deformable as the body material (eg: a fabric type material). The tail means are used to stabilise the body by placing it in operative association (although not rigidly attaching to it to) with surrounding bone in the knee joint. The tail means optionally have a cross-sectional area equal to or smaller than that of the body part, preferably at least 30% of that of the body part. A tubular gliding sleeve member of a biocompatible material is provided to the tail means to permit gliding movement of the tail means when the sleeve member is attached to bone by being placed within a bony opening, such as troughs, channels or through-extending passageways. The meniscus prosthetic device is never rigidly attached to a bone, but is positioned so that it can move in response to loadings by elongation and shortening. The device is optionally formed by wrapping a continuous length of deformable material around the tail means and a coating in the form of Dacron mesh or porous metal may be applied to all or part of the body part to permit soft tissue or bony ingrowth. The device is described to stabilising the knee against unphysiologic motions or deviations, particularly in conjunction with ligamentous reconstructions, and sustaining the compression and torsional loads normally born by the meniscus. The device is further described as serving to lubricate and aid in the metabolism of the articular surfaces of the knee joint, expanding and pressing against the soft tissue surrounding the joint and serving as an attachment to the soft tissue around the joint. No description is given as to how the necessary high level of lubrication efficiency of the meniscus prosthetic device is achieved. The material of the body part of the device is un-reinforced and, as a result, the body part would tend to be extruded from beneath the femoral condyles under heavy loadings. Suturing of the device into place and the ingrowth of fibrous tissue may compromise the necessary translational freedom of the device. Restraint of the tail means in gliding movement is unlikely to provide appropriate translational restraint of the device.

A further example is that taught by Richmond in U.S. Pat. No. 4,919,667. In this example, a soft tissue implant is created by the progressive lamination of two or more layers of felt-like Dacron® or Teflon® material between top and bottom layers of woven Dacron® cloth, the arrangement being interbonded and encapsulated in a polyurethane liquid which is cured following each step. The polyurethane liquid penetrates only partially into the layers of felted material which remain fluffy and pliable. A knitted Dacron® tube is bonded to the convex edge of the implant, extending symmetrically through 180° to 200° of arc and a narrow, high tensile strength Dacron® tape is passed through the tube with extended ends of the tape utilised for attachment of the implant. The implantation process comprises boring angled holes downward through the top of the tibia to emerge at the sides. The tapes are led down through the holes and their ends secured to the sides of the tibia. Growth of fibrous tissue over time is stated to enter the knitted fabric of the tube and firmly hold the implant in place in the joint while permitting a natural degree of sliding movement of the implant with respect to the tibia and condyle during normal flexing of the joint. Lubrication is provided by coating with body liquids present in joints. No provision appears to be made for accurate shaping and finishing of the implant upper and lower surfaces. No description is given as to how the necessary high level of lubrication efficiency of the implant is achieved. It would be difficult to control the degree of penetration of the polyurethane liquid into felted material during lamination and it is doubtful that the material of the device would properly accommodate the shock loadings normally sustained by the knee joint. It is likely that the felted material would tend to collapse over time and its initial shock absorbent capacity would diminish. Growth of natural fibrous tissue into the tube will be largely uncontrolled and may compromise the necessary translational freedom of the implant. Securing the implant in place using only the tape is unlikely to provide adequate security of translational restraint of the implant.

A further example is that taught by Mansmann in U.S. Pat. No. 6,629,997. In this example, implants to replace menisci in various forms of human joint are provided. Implants of wedge-shaped cross-sectional shape are made from a hydrogel material with smooth inner surfaces and an embedded three-dimensional mesh that reinforces the hydrogel extends beyond the peripheral rim of the implant to provide anchoring attachments. Anchoring reinforcements in the form of moulded or machined eyelets are located around the periphery of the mesh. The three-dimensional mesh (interpenetrating network of fibres) optionally incorporates a variety of long fibre types in different parts of the implant, the fibres optionally being non-resorbable and resorbable. The network of fibres is created using a variety of methods, including weaving, braiding, knitting, knotting or other comparable methods for interlacing or attaching pre-existing fibres, and is provided to mitigate the low resistance of hydrogels to tearing and cutting and to confer greater strength and durability to the implant. Implants help distribute, share and reduce the compressive loads that are imposed on the femoral and tibial segments of cartilage and may be made in a single compartment or in two compartments. Where implants are made in two compartments, they are separate from each other but connected to each other by a bundle, belt or sheet of the mesh material that is embedded in both of the hydrogel components. The three-dimensional mesh is led out to the peripheral rim of implants to provide anchoring attachments. A relatively thin layer of hydrogel material covers the embedded mesh on any “articulating” surface to minimise risk of abrasion of the cartilage surfaces on the femoral runners and tibial plateau. The outer layer is optionally made from a material different from the interior gel material, for example, allowing the inner portion of the hydrogel material (or an elastomeric material that does not retain water molecules, and which is not classified as a hydrogel) to be selected for high strength, while the outermost layer is made of a different or modified hydrogel material that is selected and designed to be highly “lubricious”. A lubricious surface can be provided by a hydrophilic polymeric material with opencell pores on its surfaces by various means, either by proper selection and treatment of the monomers that will form a polymer or by moulding in a polymeric material that has forced into a layer of granules (such as granules of salt, sugar, or similar material, that will not damage the polymer and can be rinsed out (dissolved away) when the polymer has fully set. Alternatively, the surface layer of a polymer can be provided with numerous hydrophilic molecular groups that are exposed and accessible to water molecules. Various methods are described for moulding of the implant within and around the interpenetrating network of fibres. The peripheral surface is optionally made from a tough elastomeric material, or with a porous outer layer of hydrogel to promote tissue ingrowth to the porous rim region of the implant. The various methods proposed to ensure proper lubrication of the implant (to provide a lubricious surface): natural hydrophilicity of the hydrogel material, opencell pores, or provision of numerous hydrophilic molecular groups are of doubtful efficacy. Regardless of the internal structure of the implant, hydrogels having a highly hydrophilic surface are, generally speaking, soft and would have a limited wear life. Embedding of sugar or salt granules is unlikely to provide a surface with a fine porous structure. No description is given of methods of providing numerous hydrophilic molecular groups in surfaces. It would be difficult to maintain the integrity of the complex interpenetrating network of fibres while moulding the implant within and around it. Tissue ingrowth into the porous rim region of the implant would be largely uncontrolled and might compromise the necessary translational freedom of the implant. Apart from the passing of sutures through the eyelets located around the periphery of the mesh, no description is given of the method of fixing the horns (free ends) of the implant.

A further example is that taught by Weissberg in WO 2008/127924 A1. In this example, a prosthetic meniscus is made with an outer surface made from a lubricious material, with the cross-sectional wedge shape of a natural meniscus, and with reinforcing fibres to provide durability and further mimic the characteristics of natural meniscus under stresses and loads typical of a human knee joint. The prosthetic meniscus is fitted at its free ends with elongated anchoring leads, the leads having locking anchors or threads of a resilient material to engage or lock into surgically drilled anchor holes passing across the upper part of the tibia. The anchor holes have diameters such as to just admit the locking anchors or threads which deform as they pass through the anchor holes. The anchor holes are optionally stepped and are optionally drilled down through the tibial plateau or into the sides of the tibia just beneath the tibial plateau, their points of entry being positioned at the desired locations of the free ends of the prosthetic meniscus. The anchoring leads are passed through the anchor holes, the locking anchors or threads frictionally engaging the walls of the holes or steps within the holes, thereby preventing their withdrawal and securely locating the prosthetic meniscus. Although not stated in the description, it is assumed that the anchoring leads are flexible as it would be impossible to install them if they were rigid. No description is given of the material of the prosthetic meniscus and its characteristics. Although the prosthetic meniscus is described as having a lubricious surface, no description is given as to how the necessary high level of lubrication efficiency of the prosthetic meniscus is achieved. Although the prosthetic meniscus is described as having reinforcing fibres, no description is given of their characteristics or how they are incorporated. If the prosthetic meniscus is restrained from elastic extension by the reinforcing fibres, the method of securing it with anchoring leads might compromise its necessary translational freedom. Where revision of the prosthetic meniscus is required, removal of the anchoring leads may be difficult.

A further example is that taught by Fox in WO 2008/045807 A2. In this example, a prosthetic device is provided to replace a meniscus that has been damaged, ruptured, disintegrated, diseased or is otherwise in need of replacement. The lower surface of the device is made more or less flat to comply with the surface shaping of the tibial plateau and the upper surface comprises an outer body portion in the form of a rim or wall having a height of between 5 and 15 millimetres which slopes down to a more or less flat upper surface of a central body portion having a height of between 0.1 and 5 millimetres. The upper surface of the central body portion and the taper of the upper surface of the outer body portion define a concave recess configured for receiving a portion of the femur such as the femoral condyle. The prosthetic device is essentially D-shaped with the edge of the central body portion being more or less straight. The outer body portion optionally has increased stiffness relative to the central body portion and this increased stiffness may be a result of different material properties, geometries, support features and/or other mechanisms for varying the stiffness between the central body portion and the outer body portion. A number of options are described for embedding stiffening elements in the outer body portion to limit or define the amount of its deformation. A downwardly extending fixation member is integrally moulded on the lower surface of the device adjacent and more or less parallel to the straight edge of the central body portion. The fixation member is optionally deleted, is optionally made separately and attached and optionally has other positionings and orientations. The fixation member is made with a keyhole or dovetail cross-sectional shape which is adapted to engage a complementary groove that has been surgically incised in the tibial plateau. The configuration of the fixation member is such that it does not require bone screws or other means to tighten or secure it to the tibia. The fixation member is optionally the sole means utilised to secure the prosthetic device to the tibia. The fixation member is optionally utilised in combination with a downwardly extending fixation device attached to the periphery of the prosthetic device. The fixation device is fixed to the tibia by means of a bone screw, staple of other device passing through an opening in it. The prosthetic device is optionally manufactured in various sizes or is custom manufactured for a particular patient utilising characteristics determined by medical imaging techniques. To improve lubrication of the prosthetic device, the upper surface of its central body portion is provided with one or more recesses for the accumulation of synovial fluid. A range of well known polymer materials is suggested for manufacturing of the prosthetic device, including various forms of polyurethane, polycarbonate-urethane, copolymers of silicone with polyurethanes and hydrogels. Some materials are described as having, ‘ . . . some lubricious characteristics . . . ’ and the use of surface modifying end groups is nominated for the purpose of providing thromboresistance, biostability and abrasion resistance, without the need for additional post-fabrication treatments or topical coatings. The prosthetic device is described as providing shock absorption and a desirable tribology between the femur and tibia, thereby attributing to the overall therapeutic value of the prosthetic device. Although the prosthetic device is described as being able to, ‘ . . . translate and rotate with respect to the femur and/or tibia . . . ’, attachment of the prosthetic device to the tibia using the fixation member and fixation device and encouragement of tissue growth into the bottom surface of the device would inhibit such translation and rotation and may compromise the necessary translational freedom of the device. Although some materials from which the prosthetic device is manufactured are described as having lubricious characteristics, apart from the recesses to accumulate synovial fluid, no description is given as to how the necessary high level of lubrication efficiency of the device is achieved. Employment of the prosthetic device without the fixation member and fixation device is unlikely to provide adequate security of translational restraint of the implant, particularly in combination with inadequate efficiency of lubrication. The generally bulky and rigid nature of the prosthetic device, particularly where embedded stiffening elements are employed, will preclude the use of arthroscopic implantation procedures.

A further example is that taught by Linder-Ganz, Shterling, Weissberg, Elsner and Zur in WO 2009/154847 A2. In this example, a prosthetic device is provided to replace a meniscus that has been damaged, ruptured, disintegrated, diseased or is otherwise in need of replacement. The prosthetic meniscus comprises an outer body portion having a height of between 5 and 15 millimetres and a central body portion having a height of between 0.1 and 5 millimetres. The height or thickness of both the outer body portion and central body portion are varied in parts of the prosthetic device to match the anatomical features of a patient. The prosthetic device is optionally inserted in an insertion configuration and then loaded, stretched, moved and/or otherwise transferred to an implantation configuration. In some embodiments, the transformation between insertion configuration and implantation configuration is facilitated through load bearing of the device and variations in the height or thickness of the outer and central body portions are selected to accommodate the deformation or transformation. The prosthetic device is configured for use without a fixation member or fixation device to “float” within the knee joint and not be fixed to the femur or tibia. This eliminates the need to cause permanent damage to a patient's tibia or other bone structure and facilitates removal and replacement of the prosthetic device. The outer body portion is shaped and sized to prevent unwanted expulsion of the prosthetic device from the knee joint. The outer body portion completely surrounds the central body portion and optionally has a semi-ellipsoidal shape. The outer body portion comprises a first portion which extends substantially around the majority of the outer body portion and matches the shape of a natural meniscus and a second portion, the bridge component, which joins the ends of the first portion. The increased height of the outer body portion along with the contact pressure on the prosthetic device from being positioned between the femur and the tibia prevents the prosthetic device from moving outside of the desired range of positions within the knee joint. The height or thickness of the bridge component is optionally based on the size of the femur notch and the distance to the cruciate ligaments of a patient. The size and shape of the bridge component is optionally selected to achieve an optimal pressure distribution on the tibialis plateau in order to mimic the pressure distribution of a healthy natural meniscus. The bridge component and, more generally, the outer body portion are geometrically characterised by anterior, posterior, lateral-anterior, mid-lateral and lateral-posterior angles and heights as well as sagittal and coronal radii of curvature. The outer body portion and central body portion are shaped and sized such that the prosthetic device is self-centering. That is, the shape and size of the prosthetic device itself encourages the prosthetic device to position or align itself with a desired orientation within the knee joint. The upper concave surface of the prosthetic device defines vertical and horizontal bearing components. Shaped recesses are provided in the upper surface of the prosthetic device for the accumulation of synovial fluid to limit the friction between the prosthetic device and the femur. The outer body portion optionally has increased stiffness relative to the central body portion and this increased stiffness may be a result of different material properties, geometries, support features and/or other mechanisms for varying the stiffness between the central body portion and the outer body portion. A plurality of circumferentially arranged fibres embedded in the prosthetic device acts to increase the stiffness and/or hoop strength of the outer body portion relative to the central body portion. The fibres are optionally of ultra high molecular weight polyethylene. The fibres prevent the bridge component from splaying outwardly but do not prevent it from being folded inwardly into an insertion configuration. Increased stiffness of the outer body portion relative to the central body portion may be the result of different material properties, geometries, support features and or other mechanisms. The outer body portion is optionally pre-tensioned to improve the mating fit of the prosthetic device within the knee joint. The outer body portion of the prosthetic device optionally includes a deformation control element to limit the deformation of the outer body portion and to pre-tension the device. The deformation control element may be a material property, a structural property, an additional component (including carbon fibres) and/or combinations thereof, or may take the form of a reinforcing layer that serves as the deformation control element and/or pre-tensioning element. The prosthetic device optionally has a downwardly extending fixation member on its lower surface adapted to engage a complementary groove that has been surgically incised in the tibial plateau. The fixation member is optionally utilised in combination with a downwardly extending fixation device attached to the periphery of the prosthetic device, the fixation device being fixed to the tibia. The bottom surface of the prosthetic device is optionally coated with a bioactive coating to encourage the ingrowth of natural tissue, including bone growth, to further improve fixation of the prosthetic device to the tibial plateau. A range of well known polymer materials is suggested for manufacturing of the prosthetic device, including various forms of polyurethane, polycarbonate-urethane, copolymers of silicone with polyurethanes and hydrogels. Some materials are described as having, ‘ . . . some lubricious characteristics . . . ’ and the use of surface modifying end groups is nominated to create amphipathic surfaces and for the purpose of providing thromboresistance, biostability and abrasion resistance, without the need for additional post-fabrication treatments or topical coatings. Extensive manufacturing procedures are described, particularly relating to the moulding of materials and the installation of the circumferentially arranged, embedded fibres. Extensive and complex matching procedures are described for the selection of the prosthetic device by size, shape and suchlike to suit a particular patient. Extensive surgical protocols for implantation of the prosthetic device are also described, including the application of arthroscopic surgical procedures. The prosthetic device is described as providing shock absorption and a desirable tribology between the femur and tibia, thereby attributing to the overall therapeutic value of the prosthetic device. Attachment of the prosthetic device to the tibia using the fixation device and encouragement of tissue growth into the bottom surface of the device may compromise its necessary translational freedom. Although some materials from which the prosthetic device is manufactured are described as having lubricious characteristics, apart from the recesses to accumulate synovial fluid, no description is given as to how the necessary high level of lubrication efficiency of the device is achieved. Employment of the prosthetic device without any locational means is unlikely to provide adequate security of translational restraint, particularly in combination with inadequate efficiency of lubrication. The generally bulky and rigid nature of the prosthetic device, particularly where embedded stiffening elements are employed, will minimise the possibility of employing of arthroscopic implantation procedures in its implantation. Forces applied to a shape formed from a soft polymer material and constrained only by a band of peripherally arranged fibres will be compressive in nature and are likely to result in uncontrolled distortions. It is unclear what advantage would accrue from the use of surface-modifying end groups to provide amphipathic surfaces.

A final example is that taught by Osada et al in US Patent Application No. 20080119930. In this example, an artificial meniscus is provided made based upon a hydrogel having a semi-interpenetrating network structure or an interpenetrating network structure wherein a linear polymer or a network structure composing the hydrogel is a polymer of an electrically charged unsaturated monomer and/or an electrically neutral unsaturated monomer or a crosslinked product thereof, the process of synthesising the material comprising, generally, a first step of polymerizing and crosslinking a first monomer component, of which 10 mol % or more is an electrically charged unsaturated monomer, in the presence of a solvent to thereby form a first network structure; and a second step of introducing a second monomer component, of which 60 mol % or more is an electrically neutral unsaturated monomer, into the first network structure and then polymerizing the second monomer component in the presence of a solvent to thereby form a polymer in the first network structure; or optionally, further crosslinking the polymer to thereby form a second network structure in the first network structure, wherein in the case of the second monomer component being polymerized and crosslinked, the degree of crosslinking is set to be lower than the degree of crosslinking when the first monomer component is polymerized and crosslinked and wherein the molar ratio in amount of the first monomer component to the second monomer component is from 1:2 to 1:100. No description is provided as to the morphology, implantation procedure or attachment of the artificial meniscus.

Primary objects of the present invention are to provide prosthetic menisci to replace the native human knee menisci, together with surgical procedures for removal of the natural menisci and implantation of the prostheses; the prostheses being easily implantable and able to be readily matched to the shape and dimensions of the femoral condyles; the prostheses being able to be securely located on the tibial plateaus while replicating normal meniscal translation; the prostheses providing durable working surfaces having coefficients of friction similar to those of the native menisci and the material of the prostheses having compatibility with the constituents of synovial fluid; the prostheses being capable of accommodating the stresses imposed by the knee working under all normal loads; and the surgical implantation procedures and effect of the implanted prostheses being such as to require minimal rehabilitation for each patient. A secondary object of the present invention is to provide a prosthetic meniscus which may be rapidly and efficiently implanted or revised using arthroscopic surgical techniques. A tertiary object of the present invention is to provide a soft and protective prosthetic meniscus which may be temporarily implanted during repair of the femoral or tibial articular cartilage.

According to the present invention, prosthetic menisci of correct shape are made from suitable materials in the form of hollow shells, the panels constituting the shells being reinforced to sustain the forces normally applied to menisci and their external surfaces treated to render them attractive to the lubricating constituents of synovial fluid. Prior to implantation, the menisci are collapsed by evacuation of their interiors and the menisci are folded into compact form. Access is gained to the knee compartment via minimal incisions and displacement of the tendinous and capsular tissue surrounding the joint. The natural menisci are removed by surgically severing all of their tibial and capsular attachments with careful attention to haemostasis. The prosthetic menisci are correctly positioned between the femoral condyles and the tibial plateaux and secured in place by several means. A liquid of high viscosity or a polymer material setting to a gel or solid of rubbery consistency is then injected into the interior of the menisci, inflating them to conform precisely to the external shape of the tibial plateaus and femoral condyles. Knee joint components are maintained in their correct articulated relationship during inflation of the menisci. The synovial capsule is then modified as required to fully enclose the joint, separated tissue is reinstated and the skin incisions are closed.

The various aspects of the present invention will be more readily understood by reference to the following description of preferred embodiments given in relation to the accompanying drawings in which:

FIGS. 1(a) and 1(b) are lateral, diagrammatic views of the bones of the knee during extension and flexion;

FIG. 2 is a superior, diagrammatic view of the proximal end of the tibia of the right knee;

FIG. 3 is a superior, diagrammatic, transverse cross-sectional view through the right knee just above the menisci;

FIG. 4 is a fragmentary, diagrammatic view of a meniscus partially sectioned to display its internal structure;

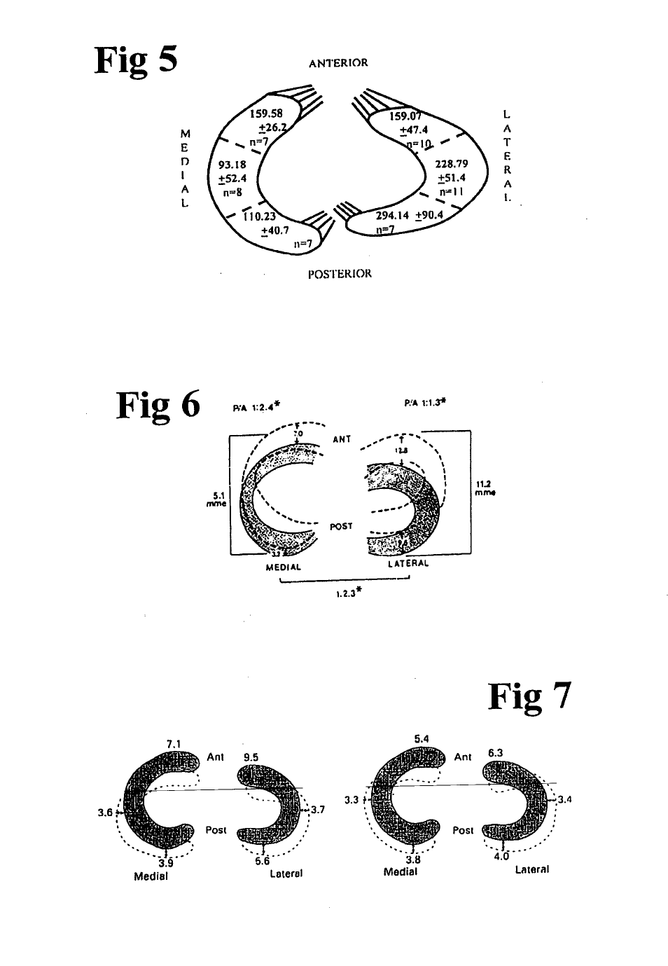

FIG. 5 is a diagrammatic presentation of regional variations of Young's Modulus in the human meniscus in tension;

FIG. 6 is a superior, diagrammatic view of displacement of menisci on the tibial plateaux at 0 and 120 degrees of knee flexion, the displaced positions depicted in solid line¹;

FIG. 7 is a superior, diagrammatic view of displacement of menisci on the tibial plateaux during 90 degrees of flexion from an erect stance with weight bearing and during 90 degrees of flexion in a sitting position, relaxed and bearing no weight, the displaced positions depicted in broken line²;

FIG. 8 is a superior, diagrammatic view of displacement of the meniscal inner margins on the tibial plateaux during deep knee flexion, the displaced margins depicted in solid line³;

FIG. 9 is a fragmentary cross-sectional view on a sagittal plane of the femoral condyle, meniscus and tibial plateau, separated to allow an appreciation of their relative dimensions;

FIG. 10 is a superior, diagrammatic view of the proximal end of the tibia of the right knee;

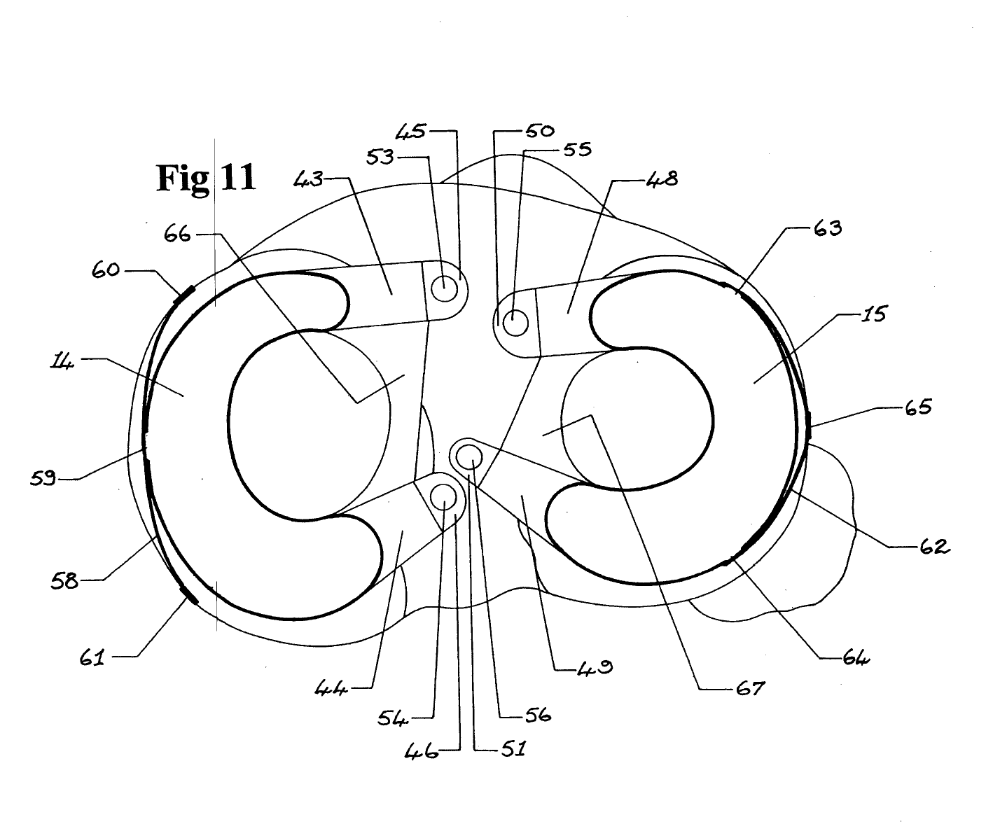

FIG. 11 is a superior, diagrammatic view of the proximal end of the tibia of the right knee depicting prosthetic menisci anchored via horns and supplementary locating means;

FIG. 12 is a diagrammatic transverse cross-sectional view of a meniscus of the present invention during moulding;

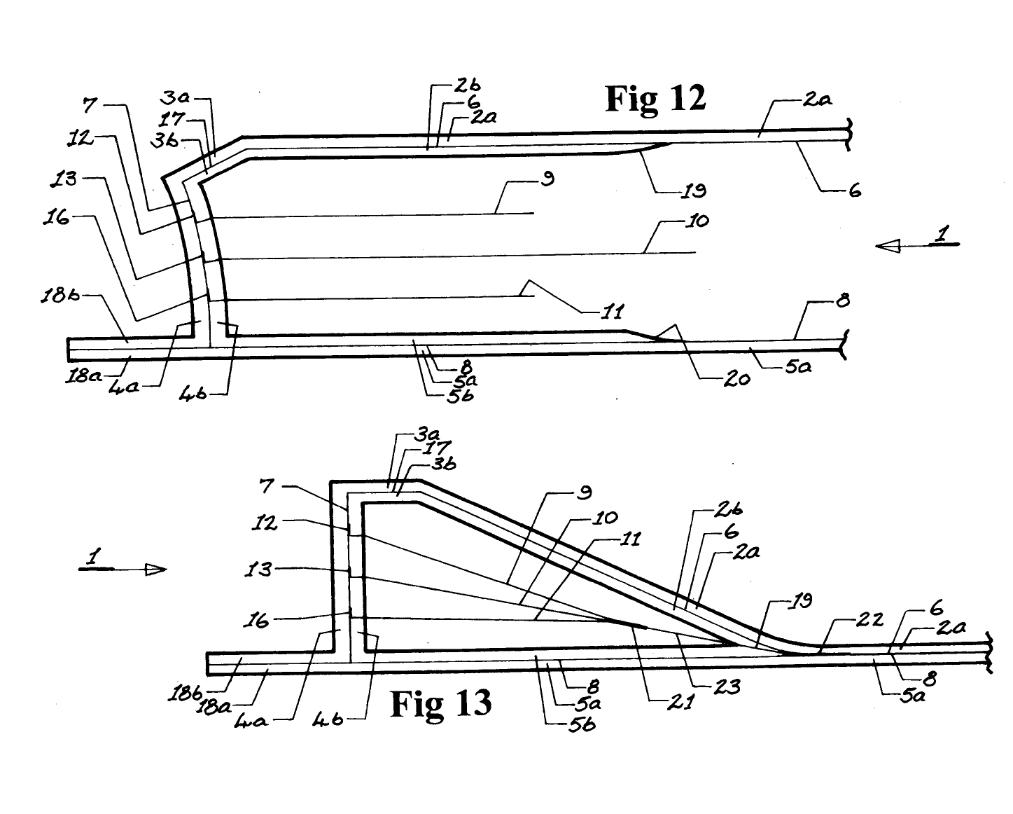

FIG. 13 is a diagrammatic transverse cross-sectional view of the meniscus of FIG. 12 after closure;

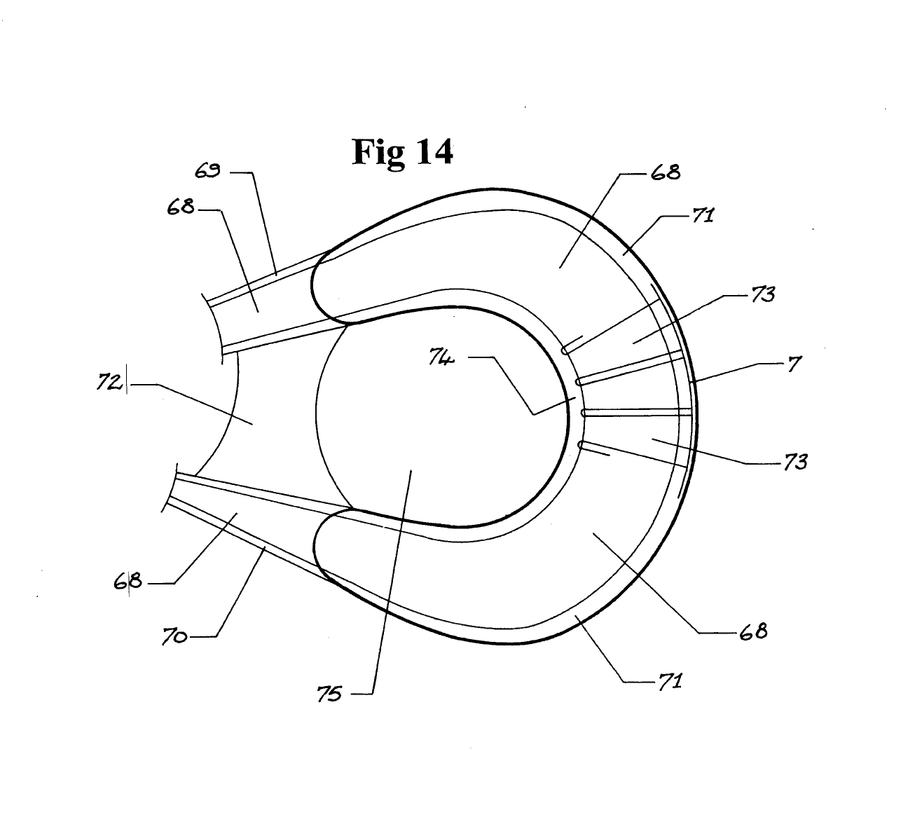

FIG. 14 is a superior diagrammatic view of a meniscus of the present invention depicting its reinforcements;

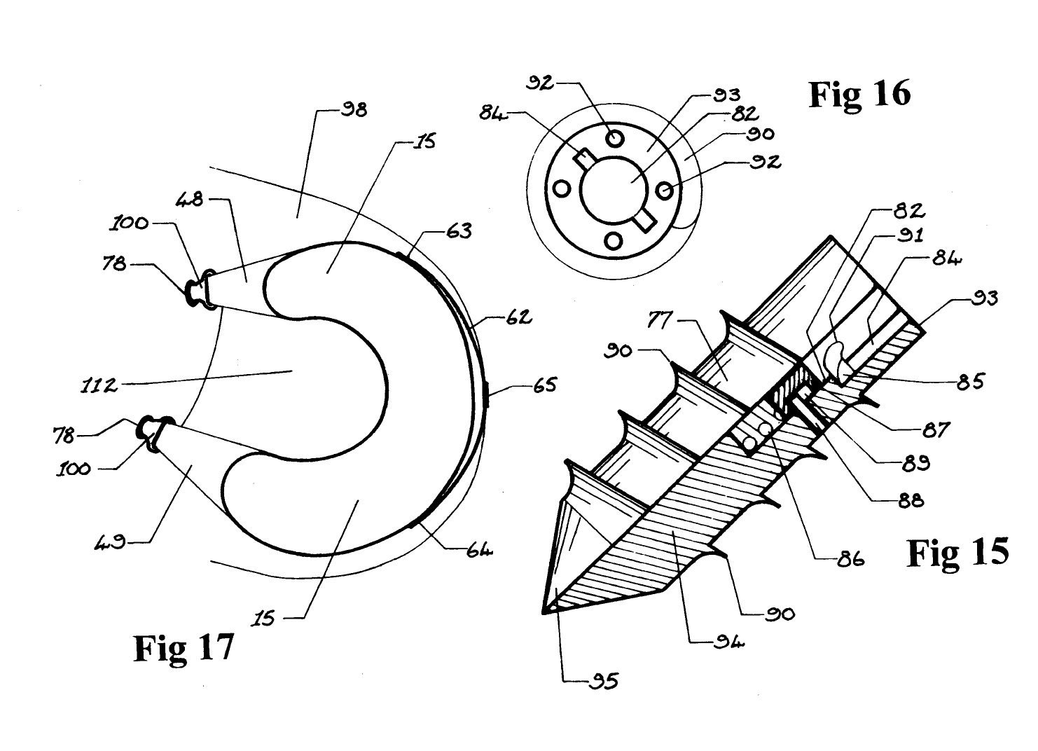

FIG. 15 is a partial longitudinal cross-sectional view of a bone anchor adapted to fixing a meniscus of the present invention;

FIG. 16 is a view of the open end of the bone anchor of FIG. 15;

FIG. 17 is a superior diagrammatic view of a meniscus of the present invention depicting its attachments;

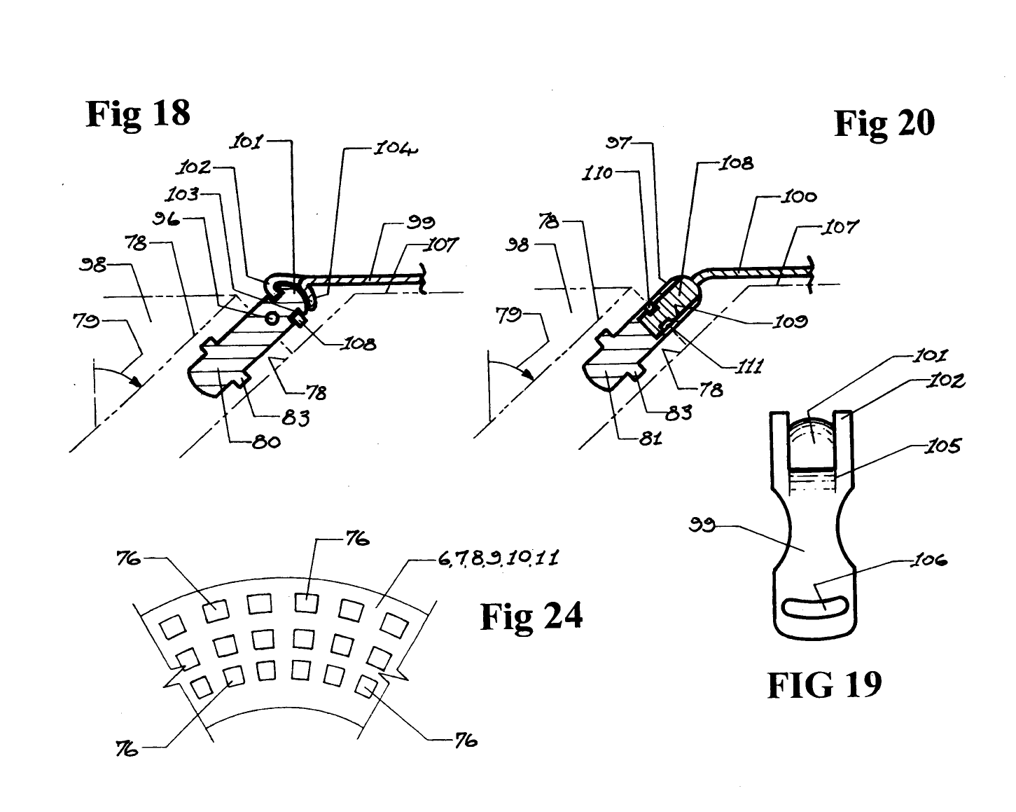

FIG. 18 is a longitudinal cross-sectional view of alternative means to anchor menisci of the present invention;

FIG. 19 is a superior view of the anchoring means of FIG. 18;

FIG. 20 is a FIG. 18 is a longitudinal cross-sectional view of alternative means to anchor menisci of the present invention;

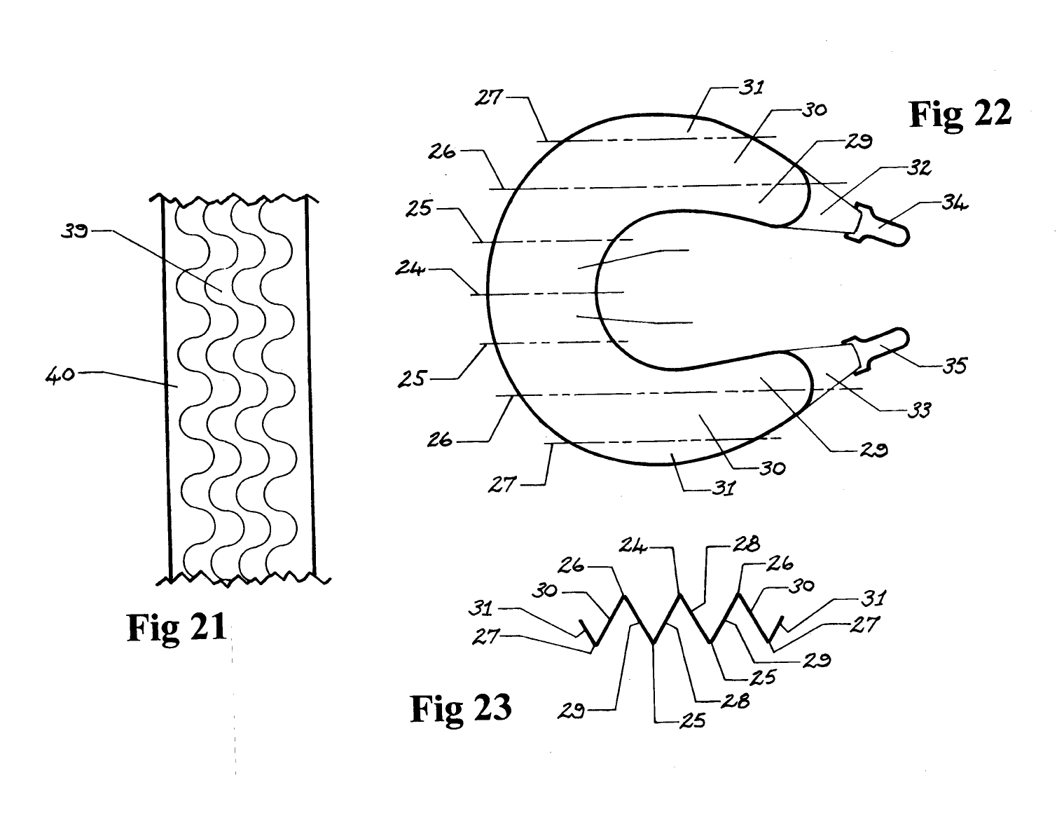

FIG. 21 is a diagrammatic face view of reinforcement means for incorporation into menisci of the present invention;

FIG. 22 is a superior view of a meniscus of the present invention depicting modes of folding prior to implantation;

FIG. 23 is a diagrammatic end view of the meniscus of FIG. 22 depicting modes of folding prior to implantation;

FIG. 24 is a fragmentary face view of reinforcement material for menisci of the present invention.

The figures are drawn to a variety of scales and no meaning or significance should be adduced from this fact.

Said prosthetic menisci are moulded from a suitable biocompatible elastomer (the base material), in the preferred embodiment, the elastomer being DSM-PTG Carbosil® 20 90A biocompatible silicone polycarbonate urethane, manufactured by DSM Biomedical, of 6167 RA Geleen, The Netherlands. The principal mechanical properties of the material are:

| Density | 1.16 | g/cc |
|---|---|---|
| Hardness, Shore A | 90 | |
| Tensile Strength, Ultimate | 42.6 | MPa |
| Tensile Strength, Yield | 6.4 | MPa |
| Elongation at Break | 530% | |
| Flexural Modulus | 0.0407 | GPa |
| Flexural Strength, yield | 1.90 | MPa |
| Tear Strength | 87.7 | kN/m |

| | |
|---|---|
| Taber Abrasion, mg/1000 Cycles | 57.0 |
| Compression Set | 15.0% |

The material combines the biocompatibility and biostability of conventional silicone elastomers with the processability and toughness of thermoplastic urethane elastomers. The material is non-cytotoxic and non-haemolytic, has a low-energy silicone surface, has outstanding oxidative stability, is hydrophobic, has high tensile strength and is optically clear.

PurSil™ silicone-polyetherurethane and CarboSil™ silicone-polycarbonateurethane are true thermoplastic copolymers containing silicone in the soft segment. These high-strength thermoplastic elastomers are prepared through a multi-step bulk synthesis where polydimethylsiloxane (PSX) is incorporated into the polymer soft segment with polytetramethyleneoxide (PTMO) (PurSil) or an aliphatic, hydroxyl-terminated polycarbonate (CarboSil). The hard segment consists of an aromatic diisocyanate, MDI, with a low molecular weight glycol chain extender. The copolymer chains are then terminated with silicone (or other) Surface-Modifying End Groups™. Aliphatic (AL) versions of these materials, with a hard segment synthesized from an aliphatic diisocyanate, are also available. PurSil and CarboSil can be melt fabricated by conventional extrusion, injection molding, or compression molding techniques. Rod, pellet, and tubing extruded from these materials displays an excellent surface finish and low gel content. In addition, these materials are heat-sealable, readily blended with fillers, and easily post-formed. In an alternative embodiment, said elastomer is Tecoflex® SG-93A thermoplastic polyurethane elastomer (polyether), manufactured by Lubrizol Advanced Materials, Inc., of Cleveland, Ohio, USA, which has a nominal Shore A hardness of 87. This material is formulated especially for solution moulding. In other alternative embodiments, elastomer materials similar in characteristics to the Carbosil and Tecoflex products and having a hardness in the Shore A range 60 to 95 are used with the present invention. In the preferred embodiment, prostheses are selected for implantation according to radiographic, ultra-sonic or magnetic resonance-derived images of the condyles. Prostheses are manufactured in a range of sizes, the small increments between successive members of a range permitting accurate matching. With reference to FIG. 5, Young's modulus (or tensile modulus) values for locations within the human menisci in tension are shown (values in MPa). It will be noted that the values are well below those of most polymer materials. For example, Kevlaa (aramid) has a tensile modulus normally in the range 83 to 186 GPa. With reference to FIG. 4, the disposition and arrangement of the natural fibrous reinforcement is depicted. The moulds for the manufacturing of said menisci are finely finished so as to provide a glass-smooth finish to the working (upper and lower) surfaces of each. In an alternative embodiment (not shown), either or both working surfaces of said menisci are provided with thin layers of a macroporous, softer and more compliant polymer material. Said thin layers preferably have a thickness in the range 0.1 to 2.0 millimetres and pores having a width in the range 50 to 300 nm. The manufacturing of polymer materials with such a porous nature is well known in the art. By providing a porous, more compliant bearing surface, this embodiment is better able to achieve microelastohydrodynamic lubrication. In another alternative embodiment (not shown), said menisci are made completely from a softer, more compliant base material. Menisci of this embodiment are employed temporarily during restoration of femoral or tibial articular cartilage and are subsequently replaced with menisci made from a harder base material.

In alternative embodiments, said prosthetic menisci are made from one or more of the synthetic polypeptide materials of the type taught by Keeley et al in Patent No. WO 2008/140703 A2⁵. These materials comprise at least three consecutive beta-sheet/beta-turn structures and at least one crosslinking amino acid residue that participates in crosslinking, wherein the crosslinking residue is distinct from the beta-sheet/beta-turn structures, each polypeptide is between 150 and 500 amino acids in length and the material is a solid or liquid. In particular aspects, each beta-sheet structure may comprise from 3 to about 7 amino acid residues. In some embodiments, the amino acid sequences of the crosslinked polypeptides are the same; while in other embodiments the amino acid sequences of the crosslinked polypeptides are different. In some embodiments, the material further comprises a reinforcing material, such as animal material, a synthetic material or metal. In other embodiments, the material further comprises a non-protein hydrophilic polymer. In some embodiments, the material further comprises glycosaminoglycan moieties, such as hyaluronan moieties. In some embodiments, the material comprises a mixture of crosslinked polypeptides and glycosaminoglycan moieties. In other embodiments, the crosslinked polypeptides are covalently linked to the glycosaminoglycan moieties. In some embodiments, the material is solid and may be in the form of pads, sheets and ligament-like structures. In other embodiments, the material is a liquid, such as a solution or suspension.

In alternative embodiments, said prosthetic menisci are made from hydrophilic polymer materials which exhibit a high degree of biocompatibility. In the preferred embodiment, said materials are hydrogels in which water absorption has been reduced and firmness increased by reduction of the proportion of hydrophilic monomers, by incorporation of hydrophobic comonomers or by an increase in the degree of crosslinking. Said prosthetic menisci are optionally made monolithic or with coatings of a similar material having different characteristics, such as greater hardness. Suitable hydrogels are those based upon methacrylate and acrylate, such as polyhydroxyethylmethacrylate; those based upon polyvinyl alcohol; those based upon polyethylene glycol, including combinations with collagen, methylated collagen or a protein such as albumin cross-linked with a collagen compound and copolymers with condensation polymers such as Nylon 6 and polyurethane (Biopol); those based upon polyethylene oxide; and those based upon acrylamide or polyacrylamide, such as polyvinylpyrrolidinone or hydrolysed polyacrylonitrile (Hypan series of hydrogels). In other alternative embodiments, modified forms of natural hydrophilic polymers, such as collagen, alginate and carrageen are employed. Other such materials are those created by derivatizing cellulose, a polysaccharide containing reactive hydroxyl groups which can easily be substituted to form ethers. Typical of these materials are sodium carboxymethyl-cellulose and hydroxyethyl and hydroxypropyl-cellulose. The properties of these polymers depend upon the molecular weight and degree of substitution. Other compounds of which copolymers and combinations are optionally employed are types I, II and IV collagen; gelatin; agarose; cell-contracted collagen containing proteoglycans, glycosaminoglycans or glycoproteins; fibronectin; laminin; bioactive peptide growth factors; cytokines; elastin; fibrin; synthetic polymeric fibres made from poly-acids such as polylactic, polyglycolic or polyamino acids, polycaprolactones or polyamino acids. Any of the preceding compounds from which said prosthetic menisci are formed are optionally reinforced with collagen microfibrils created by the various methods known in the art, including electrospinning, or by lamination with one or more thin layers of high-tensile material, as described elsewhere herein.

With reference to FIG. 11, prosthetic menisci 14, 15 are made in the manner described elsewhere herein and are free to move about on the tibial plateaus anchored, respectively, by anterior and posterior horns 43, 44 and 48, 49. In one embodiment, the ends of said horns incorporate fixing plates 45, 46, 50, 51 made from a suitable metal alloy material. Said fixing plates are located in suitable recesses (not shown) cut into the anterior and posterior intercondylar areas of the tibia and each is secured in place by one or more suitable fastenings 53, 54, 55, 56 passing through said fixing plates into the bone. In the preferred embodiment, said horns are made from a suitable biocompatible elastomer, which is optionally the same material as that from which said prosthetic menisci are formed. Said horns are strengthened by reinforcements (not shown) in the form of monofilaments or spun or braided, multi-filament yarns made from flexible materials having a suitable tensile strength. In the preferred embodiment, said reinforcements are made from Kevlar® and have a thickness in the range 0.01 to 1.0 millimetres. Said reinforcements are embedded in said horns in one or more layers and, to permit elastic extension of said horns in the range 10 to 50 percent, adopt an approximately sinusoidal form in their relaxed state. In the preferred embodiment, said sinusoidal form is characterised by combinations of wavelength and amplitude in the range 1.0 to 6.0 millimetres. In the preferred embodiment, said reinforcements of said horns pass into said reinforcements of said prosthetic menisci to form a strong connection between the two and pass through suitable apertures (not shown) in said fixing plates and double back on themselves to form a strong connection between said horns and said plates, said reinforcements being fully encapsulated in the elastomeric material of said horns. During manufacturing, said reinforcements are secured to a work surface in the desired sinusoidal arrangement with a plurality of pins and coated with an initial coating of elastic polymer material to fix the arrangement. After setting of said initial coating, said pins are removed and the coated reinforcement assembly is joined to said fixing plates and to said reinforcements of said prosthetic menisci and fully encapsulated with said elastic polymer material in a die or mould. Where said prosthetic menisci incorporate film reinforcement material, prior to said encapsulation, the end parts of said monofilament or multi-filament yarn reinforcements of said horns are woven back and forth through suitable apertures provided in the end parts of said film reinforcement material to provide a strong connection. In the preferred embodiment, said apertures in said fixing plates receiving said reinforcements are made with rounded edges to prevent chafing of said reinforcements. When loaded, said reinforcements of said horns tend to straighten, as permitted by the elastic compliance of said encapsulating polymer material, thereby providing a capacity to accommodate shock loadings.

Webs 66, 67 are optionally provided to join said horns of said prosthetic menisci, said webs transmitting hoop stresses generated in said prosthetic menisci and acting to maintain a more or less natural shaping of said menisci during the application of normal functional loadings which tend to distort them. The inner edges of said webs are preferably shaped such that, together with the inner edges of said menisci, they create roughly circular apertures. The ends of said webs are strongly attached to said horns. Said webs are reinforced by film or woven sheet material made from a strong, cross-linked polymer material with a thickness in the range 0.05 to 0.5 millimetres. The material of said reinforcements is preferably Kevlar® and arranged in the manner described elsewhere herein to provide a maximum elastic extension in the range zero to 20 percent. So as to not impede the distribution of synovial fluid, said webs are optionally made porous or foraminous.

In an alternative embodiment (not shown), said webs are deleted and the interior area of each said prosthetic meniscus is filled with an apron of thin, sheet form, the edges of which are strongly attached to said menisci and to said horns. Said apron comprises a thin, flexible film or woven sheet reinforcement encapsulated in a suitable biocompatible, elastic polymer. Said reinforcement is made from a strong, cross-linked polymer material with a thickness in the range 0.05 to 0.5 millimetres and preferably permitting elastic extension in the range zero to 20 percent. In the preferred embodiment, the material of said apron is Kevlar®. Where the reinforcement material of said apron material is not woven and therefore not porous, a large plurality of small apertures is provided in it to permit a free flow of synovial fluid through said material. In the preferred embodiment, said apertures are round or approximately round with a diameter in the range 0.25 to 3.0 millimetres with a spacing one to another in the range 0.25 to 5.0 millimetres. Also in the preferred embodiment, said apertures are arranged in patterns which create a plurality of uninterrupted stress transmission paths passing fully across the widths of said prosthetic menisci, each said stress transmission path having an angular separation from adjacent paths in the range 15° to 45°. In an alternative embodiment, said apertures are made larger and arranged to permit an elastic extension in the range zero to 20 percent.

In the preferred embodiment, the working (upper and lower) surfaces of said prosthetic menisci, said horns, said webs and said apron are treated in a variety of ways, as described elsewhere herein, to reduce friction between them and abutting biological surfaces and to improve their lubrication by synovial fluid.

Said prosthetic menisci are further located by supplementary locating straps 58, 62. Said locating straps are made with limited elastic extensibility in the range 10 to 50 percent and of a reinforced material of similar nature to that of horns 43, 44, 48, 49. Said locating straps are optionally orientated in two ways. As depicted in the figure, locating strap 58 is fixed to the outer edge of medial prosthetic meniscus 14 at a single, more or less centrally (medially) located point 59, its ends being fixed to anchors 60, 61 which are fixed to and project above the edges of the tibial plateau in antero-medial and postero-medial positions. If necessary, bone is removed from the edges of said tibial plateau to provide suitable attachment faces for said anchors. Also as depicted in the figure, the ends of locating strap 62 are fixed to the outer edge of lateral prosthetic meniscus 15 in antero-lateral and postero-lateral positions 63, 64, the central part of said locating strap being fixed to anchor 65 which is fixed to and projects above the edges of the tibial plateau in a medial position. If necessary, bone is removed from the edge of said tibial plateau to provide a suitable attachment face for said anchor. In the preferred embodiment, said reinforcement material of said locating straps passes through suitable apertures in said anchors, doubling back on itself to form a strong connection between said locating straps and said anchors, said reinforcement material being fully encapsulated in the elastomeric material of said locating straps. In the preferred embodiment, the edges of said anchor apertures are made with rounded edges to prevent chafing of said reinforcement material.

With additional reference to FIG. 21, in alternative embodiments, said horns, webs, locating straps or aprons optionally comprise one of more layers of thin, flexible film reinforcement material 39 of high tensile strength encapsulated in an elastic polymer material 40. In the preferred embodiment, said reinforcement material is Kevlar® with a thickness in the range 0.05 to 0.5 millimetres. Said reinforcement material is made with a plurality of parallel, sinusoidal cuts aligned with the designed load path. When a force is applied to said load path, said sinusoidal parts tend to straighten, as limited by the simultaneous elastic compliance of said encapsulating polymer material. Said elastic extension is preferably in the range zero to 20 percent.

With additional reference to FIG. 14, a prosthetic meniscus is made with one or more layers of reinforcement material 68 which pass fully along the length of said meniscus and along the lengths of both horns 69, 70 and are encapsulated in a suitable biocompatible elastic polymer 71. Said reinforcement material optionally takes the form of thin, flexible film or woven material of suitable tensile strength. Web 72 optionally connects said horns and is reinforced by one or more layers of said reinforcement material made contiguous with that of said meniscus proper. Said layers of reinforcement material (in said meniscus and said web) act to accommodate hoop stresses generated by normal functional loadings, said stresses tending to stretch and enlarge said meniscus and, thereby, to urge its extrusion from a joint. Closely-spaced, discrete, radially-arranged strips of similar reinforcement material 73 are distributed throughout the length of said meniscus and are preferably interleaved with said layers of reinforcement material. Said strips are preferably joined at a common band 74 which is embedded in the inner edge of said meniscus, the outer edges of said strips being fixed to the upper edge of outer panel reinforcement 7 (as depicted in FIGS. 12, 13). Said radially-arranged strips act to accommodate stresses generated by normal functional loadings which tend extrude said meniscus from the joint. In an alternative embodiment (not shown) said layers of reinforcement material are optionally extended inwardly to form the basis of an apron 75 covering the internal area of said meniscus, said apron being made continuous with said web.

With reference to FIG. 12, a prosthetic meniscus 1 of the present invention is made hollow and comprises condylar contact panel 2a, 2b, upper panel 3a, 3b, outer panel 4a, 4b and tibial contact panel 5a, 5b. Sheets of thin, flexible, polymer film reinforcement material 6, 7, 8 generally of a material of the type described herein in relation to FIG. 24 are embedded in said panels, dividing the outer (a) panel part from the inner (b) panel part. Said panels are moulded or otherwise formed from a biocompatible elastomer material of one of the types discussed in the preceding. One or more internal reinforcements (three depicted) 9, 10, 11, generally of a thin, flexible, polymer film reinforcement material of the type described in relation to FIG. 24, are optionally fixed at their outer edges along zones 12, 13, 16, respectively, to the internal surface of outer panel reinforcement 7. During manufacturing of said menisci, the outer surfaces of panel reinforcements 6, 17, 7, 8 are supported in the desired shaping by a suitable female die and the interior shapings of panel inner parts 2b, 3b, 4b, 5b are defined by a suitable male die which is made in multiple parts and divided as required to accommodate internal reinforcements 9, 10, 11; said elastomer material being injected between the adjacent surfaces of said dies. With said male die remaining in place, a larger female die defines the exterior shapings of panels 2a, 3a, 4a, together with 18b of optional extension 18a/18b and said elastomer material is injected between the adjacent surfaces of said dies. Finally, with said male die and said larger female die remaining in place, a third die defines the exterior shapings of panels 18a, 5a and said elastomer material is injected between the adjacent surfaces of said three dies. In defining the interior shapings of panels 2b, 5b, complementary shapings 19, 20 are simultaneously formed, respectively, on the distal ends of panels 2b and 5b and those parts of panel reinforcements 6, 8 distal to said complementary shapings remain exposed. With additional reference to FIG. 13, internal reinforcements 9, 11 are bonded or fused to internal reinforcement 10 along zone 21 just short of shaping 20 such that, when internal reinforcement 10 is extended under light tension and drawn down into contact with shaping 20, internal reinforcements 9, 11 are also under light tension. Said prosthetic meniscus is assembled by displacing condylar contact panel 2a, 2b inwardly to bring complementary shapings 19, 20 into abutment, that part 23 of internal reinforcement distal to zone 21 being extended under light tension and sandwiched between and bonded to said complementary shapings and bonded or fused to the exposed distal parts of panel reinforcements 6, 8 along zone 22. In an alternative embodiment (not shown), internal reinforcements 9, 10, 11 are bonded or fused together in the zone sandwiched between complementary shapings 19, 20, are bonded to said complementary shapings and are bonded or fused to exposed panel reinforcements 6, 8 in zone 22. Panels 2a, 5a, when bonded to either side of panel reinforcements 6, 8, are optionally extended to create either an annular zone or an apron covering part or all of the interior of said prosthetic meniscus. The extension 18a/18b is optionally provided as a means of attaching said prosthetic meniscus and is preferably provided only in discrete locations. Said extension is also preferably made elastic in a form similar to that of horns 43, 44, 48, 49 described in relation to FIG. 11. Panels 2a, 2b; 3a, 3b; 4a, 4b; 5a, 5b; and extension 18a/18b are made in thickness ranging from 0.5 to 5 millimetres, with different said panels being of greater or lesser thickness. With additional reference to FIG. 24, in the preferred embodiment, said thin, flexible, polymer film reinforcement material, as exemplified by any of 6, 7, 8, 9, 10, 11, is provided with a plurality of suitable apertures 76 to facilitate bonding, fusing, moulding or encapsulation of said reinforcement material by said biocompatible elastomeric polymer material, said apertures being of any suitable shape and of an arrangement such as to leave intact zones capable of satisfactorily carrying the radial and circumferential loads applied to said sheet material. In the preferred embodiment, said reinforcement material is made from Kevlar® with a thickness in the range 0.01 to 1.0 millimetres. In an alternative embodiment (not shown), said panel reinforcements and internal reinforcements are made from a woven material, said material comprising warp and weft of monofilaments, or which are optionally fused, or spun or braided, multi-filament yarn made from materials having a suitable tensile strength. In the preferred embodiment, said reinforcements are made from Kevlar® and have a diameter in the range 0.01 to 1.0 millimetres, the woven form of said reinforcements facilitating their shaping to suit the various parts of said prosthetic menisci. In the preferred embodiment, said woven reinforcements are made with a warp arranged generally radially and weft following the curved shaping of said prosthetic menisci, gaps approximately trapezoidal in shape being left between said warp and weft, said gaps having a maximum distance between opposing vertices in the range 0.5 to 5 millimetres. Following their weaving and prior to encapsulation, said woven reinforcements are coated with a thin layer of polymer material to fix the arrangement.

With reference to FIGS. 15, 16, 18, 19 and 20, in an alternative embodiment, bone anchors 77 are inserted into the anterior and posterior intercondylar areas of the tibia 98. Said bone anchors are optionally screwed into bores (indicated in broken line as 78 in FIGS. 18, 20) drilled into the bone or are made with integral cutting surfaces and self-tap into the bone. In the preferred embodiment, said bone anchors are each inclined towards the horn of the said prosthetic meniscus to be secured at an angle 79 of between 30 degrees and 60 degrees to the axis of the tibia. In a first embodiment, a said bone anchor takes the form of a cortical orthopaedic screw 77 with parallel sides on which is formed helically-arranged fluting 90. Said orthopaedic screw is made with a hollow bore 82 to receive the distal end of attachment fittings 80, 81. Said attachment fittings are secured to said bone anchor by screwing of a threaded distal part into threads (not shown) in said bore or by complementary, bayonet-type connection means. Said bayonet-type connection means comprise lugs 83 on the distal ends of said attachment fittings which pass down opposed, axial grooves 84 provided in said bore to engage short, circumferentially-arranged grooves 85. The end parts 91 of said circumferentially-arranged grooves are deflected towards the open end of said bore and act to capture said lugs against the urging of spring 86 applied to said attachment fittings via captive piston 87. Said captive piston is slideably accommodated in bore 82 and is permitted a restricted range of movement by the inner ends of one of more radially-arranged pins 88 fixed in the walls of said bone anchor intruding into said bore to engage annular recess 89 formed in said piston. Suitable apertures 92 are provided in the end 93 of said bone anchor, said apertures being engaged by a suitable tool to screw said bone anchor into bores 78 in said tibia. In the preferred embodiment, the distal part 94 of said bone anchor is made solid and said anchor is made self-tapping by the provision of one or more cutting flutings (not shown) at the tip 95. In one embodiment of said attachment fitting, suitable apertures 96 are provided at the proximal end to be engaged by a suitable tool for the purpose of engaging said attachment fitting with said bone anchor. In another embodiment of said attachment fitting, the proximal end 97 is made hexagonal or with suitable flats to be engaged by a suitable tool for the purpose of engaging said attachment fitting with said bone anchor.

In the preferred embodiment, bores 78 in said tibia are made with sufficient depth such that the proximal ends of said attachment fittings in their installed positions are more or less level with the surface 107 of said tibia. The proximal ends of said attachment fittings are connected to the horns of said menisci by means of connection pieces 99, 100. Said connection pieces are permanently attached to the ends of said horns of said menisci by said reinforcements of said horns (not shown) passing through a curved attachment slot 106 formed in the proximal end of each said connection piece. The shaping of said attachment slot ensures that tensional forces applied by said reinforcements of said meniscal horns will be applied normal to each point of contact. In a first embodiment (depicted in FIGS. 18 and 19), a said attachment fitting is provided with a circumferential groove 103 just below its head 101. The distal end 102 of said connection piece is formed into a pair of folded-back or returned, parallel claws 108 which engage said circumferential groove. A sharply folded-back, springy tab 104 formed between said claws elastically engages the rounded top of head 101 to secure said claws in place on said attachment fitting. Cuts 105 of suitable length are made in said connection piece to increase the springiness of tab 104. In a second embodiment (depicted in FIG. 20), a said connection piece is made with a cylindrical part 108 which is rotationally accommodated in bore 109 formed in the proximal part of said attachment fitting. Said cylindrical part is captured by the inner ends of one or more pins 110 fixed in the walls of said attachment fitting engaging annular groove 111 formed in said cylindrical part. In this embodiment, said cylindrical part is preferably joined to an expanded proximal end part incorporating a curved attachment slot 106 by a narrow rod 100. Where revision of said prosthetic meniscus is required, said attachment fitting is readily detached from said bone anchor by unscrewing it from said threaded bore of said bone anchor or releasing said bayonet-type connection. Said bone anchor is left permanently in place in said tibia. In the preferred embodiment, said bone anchors are made from titanium or other suitable metal alloy and are treated by one of the methods well known in the art to promote osseointegration. The embodiments depicted in FIGS. 18, 19 and 20 permit limited angular displacement of connection pieces 99, 100 across the surface of said tibial plateau.

In an alternative embodiment, said bone anchors are made in the form of cancellous bone anchors, with a tapered body parts and deeper fluting. Said bone anchors are preferably made to be self-tapping, are made from titanium and are treated by one of the methods well known in the art to promote osseointegration.

With additional reference to FIG. 17, prosthetic meniscus 15 is secured to tibia 98 by meniscal horns 48, 49 being permanently attached to the proximal ends of connection pieces 100 and the distal ends of said connection pieces being removably attached to said bone anchors (not shown) accommodated within bore 78 in said tibia. Said horns are made reinforced with a limited elastic extensibility in the range 10 to 50 percent. Said meniscus is also located by locating strap 62, of similar material and elastic extensibility as that of said horns. The ends of locating strap 62 are fixed to the outer edge of said prosthetic meniscus in antero-lateral and postero-lateral positions 63, 64, the central part of said locating strap being fixed to anchor 65 which is fixed to and projects above the edge of the tibial plateau in an approximately medial position. Bone is cut away as necessary from the edge of the tibial plateau to provide a secure seating for said anchor. Said meniscus is reinforced to accommodate the functional loads applied to it and said reinforcement is optionally extended to fill the area within said meniscus and encapsulated with a suitable biocompatible polymer material to create apron 112.

In alternative embodiments (not shown), said bone anchor is made solid and, in a first embodiment, is made at its proximal end with a rounded cap and circumferential groove similar to features 101 and 103 as depicted in FIG. 18; and, in a second embodiment with a bore similar to feature 109 as depicted in FIG. 20. In the first said embodiment, connection piece 99 engages said rounded cap and circumferential groove as described. In the second embodiment, said bore is provided with a circumferential groove and connection piece 100 has at its distal end a cylindrical part 108 incorporating a ball lock-type locking means. In said ball lock-type locking means, which are well known in the art, two or more hardened balls are kept radially displaced by a rod which is urged by a spring into place between them. Said radial displacement of said balls into said groove acts to lock said cylindrical part into place in said bore. Displacement of said rod against the urging of said spring brings a circumferential groove formed in said rod into coincidence with said balls, permitting them to withdraw from said circumferential groove and, thereby, permitting said cylindrical part to be withdrawn from said bore. The distal end of said connection piece extends into a bore in said cylindrical part and serves as said ball displacement rod. Tension applied to said connection piece applies additional force to radially displace said balls and, thereby, to lock said cylindrical part into said bore in said bone anchor. Conversely, a compression force applied to said connection piece permits said cylindrical part to be withdrawn from said bore. In the present arrangement, a suitable spring is preferably provided at the inner end of said bore to displace said cylindrical part from said bore when said balls are withdrawn.

At commencement of the implantation procedure for a particular patient, a said prosthetic meniscus and said locating straps are selected for size and shape from radiographic images. Prior to its implantation, the working (condylar and tibial contact) surfaces of said meniscus are treated as described elsewhere herein to render them attractive to the lubricating components of synovial fluid. With reference to FIGS. 22 and 23, to facilitate its implantation, said meniscus is collapsed by evacuation of its interior and folded, concertina fashion, into a compact form. The first fold is made along line 24, then along lines 25, 25; along lines 26, 26; and along lines 27, 27; thereby creating panels 28, 28; 29, 29; 30, 30; and 31, 31. The different lengths of horns 32, 33 permit attachment fittings 34, 35 to be arranged one ahead of the other during the implantation process. Access is gained to the knee compartment via minimal incisions and displacement of the tendinous and capsular tissue surrounding the joint, varus or vagus forces being applied as necessary to open the joint. The natural menisci are removed by surgically severing all of their tibial and capsular attachments with careful attention to haemostasis, a process well known in the art. Where only one said natural meniscus is removed, the transverse geniculate ligament is severed at an appropriate length and sutured to the base of the anterior cruciate ligament. Bone is removed from the anterior and posterior intercondylar areas of the tibia to accommodate said attachment lugs and from the periphery of the tibial plateaus as necessary to accommodate said locating strap anchors. Alternatively, appropriately angled bores are drilled into the anterior and posterior intercondylar areas of the tibia and suitable bone anchors are installed in said bores. In its said folded, compact form, said prosthetic meniscus is suitably lubricated, loaded into a suitably convergent positioning tube (not shown), orientated as required and extruded via a minimal incision into the knee joint. Within the knee joint, said prosthetic meniscus is unfolded and positioned appropriately between the femoral and tibial condyles and its horns are extended to permit fixing plates 45, 46, 50, 51 (as described in relation to FIG. 11) to be fixed to said tibia with suitable fastenings, or to permit paired attachment fittings 80, 99 (as described in relation to FIG. 18) or paired attachment fittings 81, 100 (as described in relation to FIG. 20) to be secured to said bone anchors installed in said tibia.

With the prosthetic meniscus correctly positioned and secured in place, a liquid of high viscosity, or liquid polymer material setting to a gel or to a solid of rubbery consistency is then injected into the interior of the meniscus, inflating it to conform precisely to the external shape of the tibial plateau and femoral condyle. Knee joint components are maintained in their correct, articulated relationship during inflation of the meniscus and gelling or setting of the inflation medium. The synovial capsule is then modified as required to fully enclose the joint, separated tissue is reinstated and the skin incisions are closed.

A wide range of biocompatible, viscous liquids or liquids settable to a gel or to a rubbery solid are suitable as inflation media and are well known in the art. Suitable viscous liquids preferably have a dynamic viscosity in the range 5 to 50 Pa·s at normal body temperature, higher viscosity liquids being employed in thinner-walled forms of said prosthetic menisci. Examples of biocompatible, viscous liquids are diglycerols, polyglycerols or silicones. An example of a biocompatible gel is the chemically gellable, aqueous, biocompatible, polysaccharide gel composition taught by Bardonnet et al in WO/2002/57355. Many other self-gelling, natural or synthetic polymers are well known. Of particular interest are the biocompatible, rapid transition polymers manufactured by Pluromed, Inc, of Woburn, Mass., USA, which can be made liquid at lower temperatures while forming a gel at normal body temperatures. As the gelling process is reversible, the ability to revert the gel to a liquid by cooling would facilitate drainage and collapse of a prosthetic meniscus in the case where revision is required. An example of a biocompatible, settable polymer is the bioactive and biocompatible polyurethane-butanediol-glycosaminoglycan copolymer taught by Masters et al in WO 2008/134468 A1. The hardness of such materials is readily manipulated by manufacturers.

Said inflation medium is preferably injected into said meniscus by means of a suitable syringe. Said injection is preferably delivered at a single site in outer panel 4a, 4b. If required, multiple injection sites in said outer panel, situated above, below and between internal reinforcements 9, 10, 11, are used. Said internal reinforcements are made generally as depicted in FIG. 24 with a large plurality of apertures throughout their areas. During injection of said inflation medium, from the evacuated and collapsed state of said meniscus, said inflation medium flows readily through said apertures in said internal reinforcements, filling all parts of the interior of said meniscus. Gelling or setting of said inflation medium acts to capture said internal reinforcements in a gel or solid matrix. In the normally loaded, functional form of said prosthetic meniscus, said internal reinforcements act to oppose any tendency for said meniscus to be extruded from a joint.

Where, because of the nature of said inflation medium, there is danger of leakage of said medium, a suitable thin, self-sealing membrane (not shown) is provided on the inner surface of said outer panel, the location of the safe injection sites so created being marked or otherwise indicated on the exterior surface of said outer panel. Upon withdrawal of the syringe from an injection site, the self-sealing nature of the membrane acts to positively close the aperture created by insertion of said syringe.

To provide improved hydrophilicity and lubrication of the working (condylar contact) surfaces of said menisci by synovial fluid, said working surfaces are treated in a variety of ways. In a first embodiment (not shown) a method⁴ is employed which renders said surfaces attractive to dipalmitoylphosphatidylcholine (DPPC) by impregnating said surfaces with poly[2-methacryloyloxyethyl phosphorylcholine-co-n-butylmethacry-late][poly(MPC-co-BMA)]. [poly (MPC-co-BMA)] is a biocompatible, lipid-attracting polymer soluble in solvent systems which also dissolve many polyurethanes. DPPC is the most abundant phospholipid in synovial fluid. In said method, the polyurethane elastomer is immersed in an ethanol solution containing BMA (0.3 mol 1⁻¹) and benzoic peroxide (1 wt % to BMA) as a polymerization initiator for 15 hours, resulting in a slightly swollen surface. The material is lightly washed with ethanol and then immersed in an ethanol solution containing MPC (0.3 mol 1⁻¹) for 30 minutes. After removal from the second solution, the material is blotted dry and then heated at 70° C. for 5 hours under an argon atmosphere to polymerize the monomers present in the surface of the material. Finally, the material is washed with ethanol and then dried en vacuo at room temperature for 24 hours. The method is adapted as required for volume application.

In another embodiment (not shown), to reduce frictional effects, the smooth polymer working surfaces of said prosthetic menisci are coated directly with a layer of diamond-like carbon (DLC) of suitable thickness. DLC is harder than most ceramics, is bioinert and has a low frictional coefficient. Where necessary, said surfaces are modified prior to deposition by ion-implantation of nitrogen, O₂ plasma treatment, or the like. In the preferred embodiment, the coating is made using pulsed laser deposition, the radio-frequency plasma CVD process or ion beam-assisted deposition, the last-named being the most preferred. Where necessary, a smooth, thin, flexible layer of a harder polymer material is bonded to said working surfaces to provide a better substrate for said coatings. In the preferred embodiment, said layer of harder material has a thickness in the range 0.05 to 0.5 millimetre, the material being a cross-linked polymer of high tensile strength, such as Kevlar®. CVD coatings have been successfully demonstrated on a wide variety of polymer materials and the above-named methods can be performed at acceptable temperatures. In an alternative embodiment (not shown), said polymer surface layers to be coated are densely impregnated with carbon nanofibres with which said DLC coating makes a strong bond.

In another embodiment (not shown), to reduce frictional effects, the smooth polymer working surfaces of said prosthetic menisci are coated with a thin layer of a wear-resistant material in the form of a suitable carbide, nitride or oxide. In the preferred embodiment, said layer is deposited from a suspended solution of nanoparticles using the electroless plating process. In a first embodiment, said surfaces are first plasma treated and a metal catalyst then infused into them by chemisorption to activate said surfaces. Typical metal catalysts are 5 nCl₂ and PdCl₂. In a second embodiment, said catalysts are infused into said surfaces using super-critical carbon dioxide (scCO₂), taking advantage of the solvency and plasticization effects of scCO₂. Where necessary, a smooth, thin, flexible layer of a harder polymer material is bonded to said working surfaces to provide a better substrate for said coatings. In the preferred embodiment, said layer of harder material has a thickness in the range 0.05 to 0.5 millimetre, the material being a cross-linked polymer of high tensile strength, such as Kevlar®.

In another embodiment (not shown), the lubricity of said working surfaces of said prosthetic menisci is enhanced by the generation thereon of a layer of hyaline cartilage. In this embodiment, a layer of suitable, highly porous scaffold material is prepared and fused or otherwise fixed to said working surfaces. In an alternative embodiment, said scaffold material is formed directly upon said working surfaces. Suitable materials for said scaffold include synthetic hydrogels created by the graft polymerization of either hydroxyethyl methacrylate (HEMA) and methyl methacrylate (MMA) or HEMA and glycol dimethacrylate (GDMA) onto soluble collagen using different cross-linking agents; polyglycolic acid (PGA) extruded and assembled into fibrous form using textile processing techniques; poly(D,L-lactide-co-glycolide) (PLG) assembled by the electospinning method; free radical polymerization of a combination of hydrolysed collagen, acrylic acid (AA) acrylamide (AAm) and distilled water and crosslinked using N,N′-methylene bisacrylamide (MBA); porous materials such as (poly)ethylene glycol-terephthalate-(poly)butylene-terephthalate (PEGT/μPBT) assembled by the controlled deposition of molten co-polymer fibres in three-dimensional form by computer-controlled syringe; or one of the other methods well known in the art. Said scaffold is seeded with articular chondrocytes and cultured in vitro under appropriate conditions. During generation of said hyaline cartilage, suitably shaped moulds are applied to the surface of said scaffold for periods to ensure appropriate surface shaping. Following generation of said hyaline cartilage, said prosthetic meniscus is implanted in the manner described herein. In an alternative embodiment, said prosthetic meniscus with layer of seeded scaffold material is implanted immediately and said hyaline cartilage is allowed to generate in-situ.

In another embodiment (not shown), said layers of cartilage are generated on the working surfaces of said prosthetic menisci using a method adapted from that taught by Kim et al in US 2010/0120149. In this embodiment, polymer scaffoldings are prepared and fused or otherwise fixed to said working surfaces and differentiated articular chondrocytes mixed with hydrogel are applied to said scaffoldings in liquid form and gelled. Said chondrocytes are then cultured in vitro under appropriate conditions. In this embodiment, said chondrocytes are first differentiated and clustered together to form cell aggregates of appropriate size using hanging drop culture, pellet culture, micromass culture, and rotational culture. Successful seeding of cell aggregates onto a polymer scaffold requires regulation of the average diameter of cell aggregates, typically in the range 10 to 800 μM. The number of cells used in formation of cell aggregates can be varied according to the type of cell used or the size of a single cell. For example, chondrocytes or bone marrow-derived mesenchymal stem cells are subjected to primary culture in the number of 1×10³ to 1×10⁷ cells, out of which 1×10³ to 1×10⁶ cells may be clustered together forming cell aggregates having an average diameter in the range 10 to 800 μM. Since the efficiency of chondrogenic differentiation is proportionate to the size of cell aggregates, it is necessary for cell aggregates to have an average diameter larger than a certain size, but not excessively larger than the pore size of the polymer scaffold, which would render it difficult to induce chondrogenic differentiation inside the polymer scaffold. The cell aggregates of differentiated chondrocytes are mixed with hydrogels in a solution state to form a cell aggregate-hydrogel complex in which the cell aggregates are evenly dispersed. The cell aggregates and hydrogels may be mixed in a weight ratio in the range of 1:1 to 1:100. This allows the establishment of a three-dimensional environment physiologically similar to that of natural cartilage. A smaller proportion of cell aggregates is unfavorable to cartilage regeneration, chondrogenesis-related ECM molecules secreted from the cell aggregates being unsuccessfully diffused and delivered, resulting in a slow progress in chondrogenesis. Suitable hydrogels for the present invention include, but are not limited to, fibrin, gelatin, collagen, hyaluronic acid, agarose, chitosan, polyphosphazine, polyacrylate, polyglactic acid, polyglycolic acid, pluronic acid, alginate, salts and the like, used alone or in mixture form. The complex of cell aggregates of differentiated chondrocytes evenly dispersed in the hydrogel matrix is applied in a solution state to a polymer scaffold and solidified into a gel state to obtain a cell aggregate-hydrogel-polymer scaffold complex. The gelation method depends upon the type of hydrogel used, the polymer scaffold providing a secure support for the hydrogel. The polymer scaffold is made from biodegradable and biocompatible polymers including, but not limited to collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethyleneoxide)-poly(propyleneoxide)-poly (ethyleneoxide)copolymer, copolymers thereof and mixtures thereof. For the successful seeding of the cell aggregate-hydrogel complex onto the polymer scaffold, the polymer scaffold should have an interconnective porous structure with a uniform pore size typically in the range of 10 to 800 μM or, more specifically, 100 to 500 μM. Additionally, the polymer scaffold requires a porosity in the range 40% to 97%. If the porosity is not more than 40%, the pore interconnectivity is remarkably reduced, while if the porosity of the polymer scaffold exceeds 97%, the mechanical strength thereof is significantly lowered. Typically, the polymer scaffold should have a porosity in the range of 50% to 97% or, more specifically, 70% to 95%. The polymer scaffold can be prepared from the biocompatible polymers detailed above using methods well known in the art including, for example, casting/solvent extraction, gas foaming, phase separation, electrospinning, gel spinning, and the like. The cell aggregate-hydrogel-polymer scaffold complex created in the manner described has a structure in which the cell aggregates of differentiated chondrocytes are evenly dispersed in the hydrogel matrix, to form a cell aggregate-hydrogel complex, and the cell aggregate-hydrogel complex is immobilized onto the surface of the polymer scaffold while simultaneously filling up the pores thereof. The cell aggregate-hydrogel-polymer scaffold complex has the following advantages: it can efficiently induce chonrogenic differentiation due to the high degree of intracellular interaction resulting from the use of cell aggregates rather than single cells; the hydrogel creates a three-dimensional environment physiologically similar to that of natural cartilage; the cell aggregate-hydrogel-polymer scaffold complex further improves the efficiency of chondrogenic differentiation; the use of a polymer scaffold enables the maintenance of high mechanical strength accurate shaping, flexibility and uniform morphology during the chondrogenic differentiation; and the cell aggregate-hydrogel-polymer scaffold complex provides cartilage tissue with high mechanical strength, flexibility, and uniform morphology. In this embodiment, said polymer scaffold is first fused or otherwise fixed to said working surfaces of said prosthetic menisci before application of said cell aggregate-hydrogel complex in liquid form. In the preferred embodiment, said polymer scaffold typically has a thickness in the range 0.5 to 3 millimetres.

In another embodiment (not shown), to provide improved hydrophilicity and lubrication of said working surfaces of said prosthetic menisci, scaffold material is fused or otherwise fixed to said working surfaces and seeded with articular chondrocytes, said working surfaces being prepared by the generation of a fluoridated hydroxyapatite coating on which is deposited a bone-like, microstructured beta-tricalcium phosphate layer. Said coating and said layer simulate the layer of calcified articular cartilage normally abutting subchondral bone. Said fluoridated hydroxyapatite coating is optionally infused into said working surfaces using super-critical carbon dioxide (scCO₂), taking advantage of the solvency and plasticization effects of scCO₂.

In another embodiment (not shown), to improve the distribution of synovial fluid between the bearing surfaces of said prosthetic menisci and the femoral and tibial articular cartilage, a network of narrow channels is moulded into one or both said bearing surfaces. In the preferred embodiment, said channels have a width of between 0.25 and 2.0 millimetres, a depth of between 0.25 and 2.0 millimetres, have a part-spherical or other suitable cross-sectional shape, are separated by between 1.0 and 5.0 millimetres and are orientated more or less radially and circumferentially. Also for the same purpose, either or both said bearing surfaces are provided at some or all of the points of intersection of said channels with recesses orientated more or less normal to the surface at each point, having a depth of between 0.5 and 5.0 millimetres and a diameter of between 0.5 and 5.0 millimetres. For the same purpose, either or both said bearing surfaces are provided with recesses orientated more or less normal to the surface at each point, said recesses having a depth of between 0.5 and 5.0 millimetres, a diameter of between 0.5 and 5.0 millimetres and being separated from each other by a distance of between 0.5 and 10 millimetres. For the same purpose, said prosthetic menisci are provided with a plurality of ducts passing from said lower bearing surface to said upper bearing surface, said ducts being orientated more or less normal to said lower bearing surface, having a diameter of between 0.5 and 5.0 millimetres and being separated from each other by a distance of between 0.5 and 10 millimetres.

In another embodiment (not shown), to provide improved hydrophilicity and lubrication of said working surfaces of said prosthetic menisci, hyaluronic acid is introduced into said polymer material from which said prosthetic meniscus is made or is applied to its working surfaces using methods adapted from those taught by Kim et al in U.S. Pat. No. 4,500,676. In a first method, hyaluronic acid is dissolved in an appropriate solvent and the obtained solution mixed with a polymer solution or an emulsion. An article is formed from the mixture or the mixture is applied as a coating. Suitable solvents for dissolving hyaluronic acid are water, dimethylsulfoxide and dimethylformamide. The surface of the polymeric substrate may be activated, as can the hyaluronic acid. In a second method, hyaluronic acid is added in a solid form, preferably as a powder, to a polymer solution. An article is formed from the mixture or the mixture is applied as a coating. In a third method, a particulate material is coated with hyaluronic acid from solution and the coated particles are introduced into a polymer solution. An article is formed from the mixture or the mixture is applied as a coating. Examples of suitable particulate materials are ion exchange resins, silica, alumina, etc. In a fourth method, in any of the embodiments described, the hyaluronic acid can be cross-linked before or after mixing with the polymer using various cross-linking agents. A typical cross-linking agent is the polymer CX100.

In another embodiment⁶ (not shown), to provide improved hydrophilicity and lubrication of said working surfaces of said prosthetic menisci, an ultrathin layer of polysaccharide is generated on said surfaces by silanizing said surfaces and applying to them a derivatized polysaccharide created by conjugating a silane alkylic chain with hyaluronan. Said surfaces are preferably first modified by corona discharge followed by application of an organo-functional silane. Alternatively, UV treatment in the presence of allylsilanes is used to introduce Si—H groups onto said surfaces.

In another embodiment (not shown), to provide improved hydrophilicity and lubrication of said working surfaces of said prosthetic menisci, using a method adapted from those taught by Swan et al in U.S. Pat. No. 5,414,075, Swan in U.S. Pat. No. 6,278,018 and Chappa et al in U.S. Pat. No. 7,736,689, a grafting reagent is used to form a polymeric layer on a support surface, and particularly on a porous support surface, in a manner that provides and/or preserves desired properties (such as porosity) of the surface. The reagent and method can be used to provide a thin, conformable, uniform, uncrosslinked coating having desired properties onto the surface of a preformed, and particularly a porous, polymeric substrate. The method includes the steps of, a) providing a porous support surface, b) providing a nonpolymeric grafting reagent comprising a photoinitator group, c) providing one or more polymerizable monomers adapted to be contacted with the surface, in the presence of the grafting reagent, and to be polymerized upon activation of the photoinitiator and, d) applying the grafting reagent and monomer(s) to the surface in a manner, and under conditions, suitable to coat the surface with the grafting reagent and to cause the polymerization of monomers to the surface upon activation of the grafting reagent. The following laboratory procedure is adapted for the surface modification of polyurethane (PU) by application of acrylamide/acrylamidomethylpropane sulfonic acid (AMPS) with the restrained, multifunctional reagent for surface modification (RMRSM) as taught by Swan et al. RMRSM was prepared according to the method described in Example 1 as taught by Swan et al. A coating solution was prepared by dissolving an amount of RMRSM at 1 g/l in 100% isopropyl alcohol (IPA). Polyurethane rods (5 cm (2 in.) long, Thermedics) were wiped with an IPA (99% purity) soaked lint-free cloth and allowed to dry. The clean PU rods were then, dipped into the RMRSM solution, previously prepared as described above, removed from the solution at a steady rate (approximately 2 cm/sec), and allowed to dry for at least 5 minutes. After applying RMRSM to the rods, the rods were placed in a solution containing a mixture of monomers (acrylamide 3% or 7% and AMPS 7% or 3% respectively, weight to volume) in deionized (DI) water. Approximately 8 ml of the monomer mixture was placed in a glass syringe (10 ml, Micro-mate™ interchangeable hypodermic syringe with lever lock, Popper and Sons, Inc.) containing a stopcock in the bottom to prevent the solution from draining out. The PU rods were placed in the syringe containing the monomer solution and nitrogen gas was allowed to bubble up into the solution for at least 10 minutes to remove oxygen in the solution. After deoxygenating, the solution containing the PU rods was exposed to UV light (EFOS light—Ultracure 100 SS Plus systems with light guide, EFOS USA Inc. in the 320-500 nm wavelength range for 150 seconds). The intensity of the light, as measured with a radiometer (International Light, IL1400A with SEL005/NS335/W), was approximately 20 mw/cm.sup.2 in the 330-340 nm wavelength measured at a distance of 2.5-3.0 cm from the end of the light source. After exposure to the UV light, the samples were removed from the monomer solution and washed thoroughly to remove any unbound residual monomer. The following laboratory procedure is adapted for the surface modification of polyurethane (PU) by application of acrylamide/acrylamidomethylpropane sulfonic acid (AMPS) with the surface coating agent (SCA) as taught by Swan. A reagent of the structure shown as Compound II was prepared in the manner taught by Swan. A coating solution was prepared containing 5 mg/ml of Compound II in DI water. PU rods (5 cm, Pellethane, EG-60D, Thermedics) were cleaned with WA (>99% purity) using a lint-free cloth and allowed to dry. The clean rods were placed in a clear glass tube containing the Compound II solution. The rods were incubated in the solution at room temperature for approximately five minutes. Following incubation, the substrate in the Compound II solution was illuminated with a Dymax flood lamp (model no. 2000 EC, Dymax Corporation, Torrington, Conn.) which contained a doped mercury vapor lamp, to activate the photoreactive groups present in Compound II, thereby attaching it to the rod surface as a base coat. The rods were illuminated for three minutes at an intensity of 1-1.5 mW/cm.sup.2 in the wavelength range of 330-340 nm at the rod position. After UV curing, the rods were rinsed in DI water for approximately 30 seconds prior to graft polymerization. Following the coating of the rods with the Compound II base coat, the rods were placed in 8.0 ml of a mixture of acrylamide (0-10%, Aldrich) and AMPS (0-10% AMPS 2405 monomer, salt solution, Lubrizol) contained in a 10 ml glass syringe (Micro-mate interchangeable hypodermic syringe with leur lock, Popper and Sons, Inc.) The monomer mixture and the substrate were then deoxygenated using nitrogen gas bubbling up from the bottom of the syringe for 10 minutes. After 10 minutes of sparging the monomer solution with nitrogen, an EFOS TV light was placed at the top of the syringe. The solution was illuminated with the EFOS light while nitrogen gas was still bubbling up through the monomer solution. The solution was illuminated for 150 seconds at an intensity of 10 mW/cm.sup.2, as measured with a Radiometer (International Light, IL1400A with SEL005/NS335/W), in the 330-340 nm wavelength at a distance of 3.0 cm from the end of the light guide. After exposing to the UV illumination, the rods were removed from the grafting solution and washed in DI water to remove any unbound monomer. The cited patents describe methods of applying other coatings to other substrates. The two methods described produce a durable, lubricious, biocompatible coating and the preservation of surface porosity ensures the maintenance of natural lubrication functionality.

In another embodiment (not shown), to provide improved hydrophilicity and lubrication of said working surfaces of said prosthetic menisci, said surfaces are functionalized and bioactive agents conjugated thereto using an atmospheric glow plasma discharge using methods taught by Roy and Raja in WO 2007/008755 A2. According to one embodiment, flow-through atmospheric pressure glow (“APG”) plasma discharges are used to functionalize polymer surfaces. Advantages of APG discharges include the (1) highly non-equilibrium chemical and thermal property of the plasma (similar to classical low-pressure glow discharges), (2) high degree of uniformity over large areas and volumes (without constriction and the resulting streamer or arc formation), (3) relatively low ion energetics and (4) one-atmosphere operation. Surface functionalization of a polymeric material may be used, among other things, to prepare the polymeric surface to accept drugs, vaccines, and/or contrast agents. Functionalization may involve the addition of positive or negative charges or charged radical groups to the surface of the particle. Drugs or contrast agents may be attached to the positive or negative charges or charged radical groups, and the polymeric material may then be used as delivery vehicles for the attached drugs or agents. The material to be functionalized is typically a polymeric material. In particular embodiments, the polymeric material is a biopolymer. In some embodiments, the biopolymer is a biodegradable polymer. Biodegradable polymers include, without limitation, PMMA, poly(lactide-co-glycolide) (PLGA), polylactic acid (PLA), polyglycolic acid (PGA), polycaprolactone (PCL), or polyethylene glycol (PEG). In a particular embodiment, the material can be a two- or three-dimensional surface such as, without limitation, a polymeric biomaterial for prostheses, or other polymer. In an alternate embodiment, the polymeric material used in non-biomedical applications may be non-polymer. Bioactive agents are attached to the functionalized polymeric surface include, without limitation, a small molecule drug, a protein drug, a peptide drug, a DNA drug, a RNA drug, an oligonucleotide drug, an immunomodulatory agent, a vaccine antigen or a contrast agent. According to one embodiment, the material to be functionalized is suspended in a feed gas that is then introduced to the plasma discharge volume. The surface of the material is functionalized as it passes through the APG plasma discharge, charged and radical species in the plasma interacting with the material and functionalizing its surface. Trace impurity species in the feed gas, such as trace amounts of oxygen, may also contribute to negative charge deposition on the polymer material surface. The APG plasma is sustained in a base working, inert gas that comprises helium, argon, nitrogen, or another diluent gas, diluent gases being preferred as base working gases as they normally support the most stable and uniform plasma discharges. When the working gas is a pure diluent working gas, pure negative charges are deposited on the surface of particles exposed to the plasma discharge. According to certain embodiments of the present invention, the base working gas may be modified by adding a small amount (e.g., about a few percent by volume) of another gas, the additive gas creating a reactive environment for processing the particles but not affecting the stability of the plasma. When plasma is sustained in a mixed gas, radical groups and electrical charges are deposited on the surface of particles exposed to the plasma. Examples of mixed gases that yield a positive charge deposition include nitrogen with ammonia (NH₃) additive and helium with ammonia additive. A positively charged functionalized surface is used to conjugate negatively charged agents such as nucleic acids. The APG plasma efficiently decomposes NH₃ to produce molecular and atomic fragments including NH₂, NH, N, H, and their corresponding ionic form and these “daughter” radical species can subsequently be transported to the particle or surface to be functionalized, and functionalize the surface to form, for example, grafted amine groups. Other examples of additive gases are oxygen and fluorine. The APG plasma is generated in any of several configurations. In one embodiment, the APG configuration involves a dielectric-bather discharge (“DB-APG”). A DB-APG may be generated by arranging two parallel electrodes in close proximity, usually a few millimeters apart, one or both of the electrodes being covered by a dielectric layer, such as, for example, polycarbonate. The electrodes are driven by a high voltage power supply at high audio, for example, voltage of approximately 1 kV and audio of approximately 10 kHz are appropriate. The APG plasma is operated within a narrow range of parameters to create uniform and stable plasma discharges that are not unduly disrupted by APG plasma-surface interactions, the presence of a surface sometimes significantly affects plasma stability boundaries and greatly increase the propensity for plasma constriction and streamer filamentary arc formation. Plasma stability boundaries may be affected by a number of factors, such as surface characteristics, and other plasma operating parameters known to those skilled in the art. According to certain embodiments, electrical characterization may be used to evaluate the plasma. Electrical characterization is based on discharge voltage-current measurements, the shape and magnitude of current waveforms aiding in the prediction of important plasma properties, such as the plasma mode (Townsend versus glow mode), plasma intensity, and the propensity for glow-to-arc transitions. Also, changes in the plasma state during processing are monitored through comparison of the discharge voltage-current waveforms of well-studied non-reactive APG plasma with APG plasmas in mixtures of gases and in the presence of a surface. Furthermore, waveform characteristics such as random current pulse formation, which are indicators of glow-to-arc transitions, may be useful to establish stability boundaries for the APG plasma in the processing environment. Electrical characterization may also yield insights useful for scale-up in a manufacturing setting. According to certain embodiments, time-resolved optical imaging may allow direct observation of the APG plasma structure during materials processing. An intensified charge coupled device (ICCD) camera system with about a 768×494 pixel resolution and an intensifier that can be gated down to about 100 ns at a framing rate of about 1 kHz may be satisfactory to provide time-resolved images of the discharge. ICCD imaging of an APG plasma under processing conditions may provide evidence of the impact of particle processing on the structure and uniformity of plasma.

According to certain embodiments, optical emission spectroscopy may provide evidence of the chemical structure of the APG plasma. Line emission from atomic species and band emission from molecular species may be identified and used to detect the species' absence or presence in the discharge. Line and band emission may also indicate the relative densities of the species under different discharge conditions. Through proper calibration and detailed measurements of the line and band shapes, additional information such as the absolute number densities of species, electron temperatures, and gas temperatures may also be estimated. The relative intensities of these emission bands under different processing conditions may be monitored to infer the relative densities of these species and correlated with the attributes of the surface functionalization. A time-resolved spectra from the plasma may be obtained using a 0.25 m spectrograph with a gated intensified linear diode array detector. In this way, light may be collected from the centre of the discharge and transferred to the spectrograph using, for example, a 100 micron core optical fiber. In the present case, the functionalized surfaces are subsequently loaded with hyaluronic acid, DPPC or the like. Plasma surface modification methods are well known in the art and, in alternative embodiments, other methods are employed to functionalize the working surfaces of said prosthetic menisci.

In another embodiment (not shown), where the material from which said prosthetic menisci are made is not fully a crystalline polymer, the lubricity of said working surfaces of said prosthetic menisci is enhanced by the impregnation of the surface with hyaluronic acid or other lubricating component of synovial fluid. In this embodiment, impregnation takes place after fabrication and is effected by dispersing said lubricating component in finely divided dry form over the surface to be treated and subjecting said meniscus to supercritical carbon dioxide at a temperature above 30.1° C. and pressure in the range 150 to 180 bar. Said lubricating component is dissolved by the super-critical carbon dioxide and diffused into said surface Satisfactory impregnation is normally achieved in less than three minutes.

In another embodiment (not shown), the lubricity of said working surfaces of said prosthetic menisci is enhanced by providing means to trap a layer of the lubricating components of synovial fluid on the working surfaces of said prosthetic meniscus. In this embodiment, said trapping is effected by the provision of a dense layer of electrospun fibres on said working surfaces. In the preferred embodiment, said electrospun fibres are spun from molten polymer precursors and are entrained in a flow of heated gas at a temperature in the range 0 to 25 percent higher than the glass transition temperature of the spun fibres. Said spun fibres impact a said working surface being treated and fuse at the point of contact, the greater part of said fibres remaining unattached to said working surface.

In another embodiment (not shown), the lubricity of said working surfaces of said prosthetic menisci is enhanced by the generation of polymer brushes on said surfaces. While many techniques have been developed for the synthesising of polymer brushes, in the preferred embodiment, said brushes are grown using surface initiated polymerisation (SIP) or atom-transfer radical polymerisation (ATRP) with the initiator molecule transferred to the substrate using microcontact printing. In alternative embodiments, other methods of synthesising polymer brushes are employed.

In another embodiment (not shown), the lubricity of said working surfaces of said prosthetic menisci is enhanced by making the material of said prosthetic menisci with appropriate surface modifying end groups. Surface active endgroup-containing polymers and their preparation are well known in the art. In the preferred embodiment, surface modifying end groups are incorporated into the material from which said prosthetic menisci are made render said working surfaces hydrophilic and attractive to the lubricating constituents of synovial fluid.

In another embodiment (not shown), the lubricity of said working surfaces of said prosthetic menisci is enhanced by a method adapted from those taught by Ward in EP 2 213 293 A2 for the immobilization of biologically-active entities, including proteins, peptides, and polysaccharides, at a surface of a polymer body. The use of surface modifying endgroups (SMEs) to tailor polymer surface properties is known (see U.S. Pat. No. 5,589,563 and US 2005/0282977 A1: Ward et al). A variety of simple hydrophobic and hydrophilic endgroups has been demonstrated to enable the achievement of useful changes in surface properties of polymers. Such surface properties include biostability, protein adsorption, abrasion resistance, bacterial adhesion and proliferation, fibroblast adhesion, and coefficient of friction. Polymers of the types disclosed in the said prior art references may be used as base polymers for carrying the covalently bonded Self-Assembling Monolayer endgroups of the present invention. A “self-assembling moiety”-containing polymer molecule endgroup is defined as an endgroup that spontaneously rearranges its positioning in a polymer body to position the moiety on the surface of the body, which positioning effects a reduction in interfacial energy. The endgroup structure may comprise one or more chemical groups, chains, or oligomers that spontaneously assemble in the outermost monolayer of the surface of the polymer body, or may comprise one or more chemical groups, chains, or oligomers that spontaneously assemble within the bulk of the polymer body. The polymer bulk is defined as the region within the polymer body that is at least one monolayer away from the outermost monolayer of the polymer body surface. The method provides for the configuration of the nanostructure, supramolecular structure, and/or conformation of a molecular monolayer at a surface of a polymer body at an interface. It involves contacting the polymer body surface with a separate medium to form an interface under conditions that facilitate the delivery of endgroup molecular moieties to the polymer body surface and maximize the resulting concentration of head groups in the outermost surface. This delivery is, in part, due to the interaction of chemical groups, chains, or oligomers in the endgroup moieties. The endgroup molecular moieties are covalently or ionically bonded to a polymer in the body and include one or more chemical groups, chains, or oligomers that spontaneously assemble in the outermost monolayer of the surface of the polymer body or one or more chemical groups, chains, or oligomers that spontaneously assemble within that portion of the polymer body that is at least one monolayer away from the outermost monolayer of the polymer body surface. In accordance with the method, the endgroups are bonded to the polymers through a divalent oligomeric chain, having at least 5 repeat units, that is capable of self-assembly with corresponding chains on adjacent molecules of the polymeric composition. Suitable structures for the spacer chains can be found in the SAM and silane literature. In general, self-assembling spacer chains suitable for polymer endgroups of the present invention will be those that self assemble when present in self-assembling thiol or silane SAMs. The surface-modifying endgroup moieties may be delivered to the polymer body surface by their spontaneous diffusion to the surface region of the polymer body or by their rearrangement or repacking in the surface layer of the polymer body. The polymer comprising the surface-modifying endgroup moieties in the polymer body makes up the entirety, or a major portion, of the body and has a weight average molecular weight in the range 5000-5,000,000 daltons, preferably in the range 50,000-1,000,000 daltons. Optionally, delivery of surface-modifying endgroups to the polymer body surface can be accomplished by adding a Surface-Modifying Additive (SMA) to the polymer just described, with the additive comprising a second polymer that is covalently or ionically bonded to the surface-modifying endgroup moieties of the present invention. When delivery of the surface-modifying endgroup moiety to the polymer surface is accomplished by adding an SMA to the polymer to be modified, the useful molecular weight range of the polymer used as an SMA may be lower: 1000-5,000,000 daltons and preferably in the range 5000 to 200,000 daltons. This is because the SMA is typically used in low bulk concentrations, e.g. less than 15 weight-%, and preferably about 1 to 5 weight-%, so that the physical-mechanical properties of the base polymer/SMA blend will be largely determined by the base polymer being modified. A very low SMA molecular weight may cause the SMA to be fugitive from the polymer being modified, e.g. by leaching or even volatilizing from the surface of the base polymer in use, particularly when there is exposure to fluids, vacuum, and/or high temperatures in use. Candidate SMA polymers with molecular weight less than 5000 are generally unsuitable and must be tested for their permanence in the base polymer before use in applications. Alternatively, delivery of surface-modifying endgroup moieties to the polymer body surface or other substrate to be modified may be accomplished by coating, plasma treatment, painting, or otherwise topically treating the surface of a pre-formed body with a material comprising a second polymer covalently or ionically bonded to the surface-modifying endgroup moieties of the present invention. The synthesising of self assembling monolayers is well known in the art and, in alternative embodiments, other methods are employed in treating said working surfaces of said prosthetic menisci.

In another embodiment (not shown), the lubricity of said working surfaces of said prosthetic menisci is enhanced by application of a method taught by Smith in U.S. Pat. No. 6,221,108 in which the polyurethane material of said working surfaces are treated with Ringer's solution in a heated bath for at least 96 hours at a temperature of between 30° C. and 37° C.

In another embodiment (not shown), the lubricity of said working surfaces of said prosthetic menisci is enhanced by making said surfaces from a semi-interpenetrating or interpenetrating network of at least two materials, one material to provide the characteristic of toughness and one to provide the characteristic of lubricity. In this embodiment, methods taught by Myung et al in US Patent Application No. 20100010114 are employed. One embodiment includes compositions of a water-swellable interpenetrating polymer network (IPN) or semi-IPN of a hydrophobic thermoset or thermoplastic polymer and an ionic polymer, the compositions exhibiting a lower coefficient of friction than the hydrophobic thermoset or thermoplastic polymer. The IPN or semi-IPN is more water-swellable, exhibits higher resistance to creep, and/or exhibits a higher conductivity and permeability than the hydrophobic thermoset or thermoplastic polymer. In some embodiments, the IPN or semi-IPN is formed by diffusing an ionizable monomer precursor solution into the hydrophobic thermoset or thermoplastic polymer and polymerizing the monomers to form the ionic polymer. In some embodiments, the composition also includes water, which may form a hydration gradient from a first portion of the composition to a second portion of the composition. An electrolyte may be dissolved in the water. The IPN or semi-IPN may also be negatively charged. In various embodiments, the hydrophobic thermoset or thermoplastic polymer is optionally physically entangled or chemically crosslinked with the ionic polymer. In some embodiments, the hydrophobic thermoset or thermoplastic polymer has ordered and disordered domains, and the ionic polymer may be disposed in the disordered domains. In various embodiments the hydrophobic thermoset or thermoplastic polymer may be selected from the group consisting of polyurethane, polymethyl methacrylate, polydimethylsiloxane, and acrylonitrile butadiene styrene. The ionic polymer may be, for example, a poly(acrylic acid) or poly(sulfopropyl methacrylate), combinations, or derivatives thereof. The ionic polymer may include carboxylate groups and/or sulfonate groups. In the preferred embodiment, the ionic polymer is hyaluronic acid. In some embodiments, the ionic polymer forms a concentration gradient from a first portion of the composition to a second portion of the composition, the concentration gradient optionally providing a stiffness and/or hydration gradient within the composition. Some embodiments include a second hydrophobic thermoset or thermoplastic polymer which is optionally disposed in a layer separate from the first hydrophobic thermoset or thermoplastic polymer or may be diffused throughout the first hydrophobic thermoset or thermoplastic polymer. Another embodiment of the invention provides a process for producing a water-swellable IPN or semi-IPN from an hydrophobic thermoset or thermoplastic polymer including the following steps of: placing an ionizable monomer solution in contact with a solid form of the hydrophobic thermoset or thermoplastic polymer; diffusing the ionizable monomer solution into the thermoset or thermoplastic polymer; and polymerizing the ionizable monomers to form a ionic polymer inside the thermoset or thermoplastic polymer, thereby forming the IPN or semi-IPN. Some embodiments include the step of swelling the IPN or semi-IPN with water, for example, to form a hydration gradient from a first portion of the composition to a second portion of the composition. The method may also include the step of swelling the IPN or semi-IPN with an electrolyte solution. Various embodiments include the steps of chemically crosslinking or physically entangling the hydrophobic thermoset or thermoplastic polymer with the ionic polymer. In embodiments in which the hydrophobic thermoset or thermoplastic polymer has ordered and disordered domains, the method may include the step of swelling the disordered domains with the ionizable monomer solution prior to the polymerizing step. In some embodiments, the hydrophobic thermoset or thermoplastic polymer is selected from the group consisting of polyurethane, polymethyl methacrylate, polydimethylsiloxane, and acrylonitrile butadiene styrene. The ionizable monomer solution may be an acrylic acid solution and may comprise monomers with carboxylate groups and/or sulfonate groups. In some embodiments, the method includes the step of foaming a concentration gradient of the ionic polymer within the IPN or semi-WN through regioselective diffusion of the ionizable monomer solution through the hydrophobic thermoset of thermoplastic polymer to, for example, provide a stiffness and/or hydration gradient within the composition. Some embodiments of the method may include, prior to the polymerizing step, the steps of placing the ionizable monomer solution in contact with a solid form of a second hydrophobic thermoset or thermoplastic polymer; and diffusing the ionizable monomer solution into the second hydrophobic thermoset or thermoplastic polymer. In such embodiments, the second hydrophobic thermoset or thermoplastic polymer may be in a separate layer adjacent to the first hydrophobic thermoset or thermoplastic polymer or may be diffused within the first hydrophobic thermoset or thermoplastic polymer. Some embodiments include the step of changing the IPN or semi-IPN from a first shape to a second shape, such as by heating the IPN or semi-IPN. Some embodiments have a second hydrophobic thermoset or thermoplastic polymer adjacent to the first hydrophobic thermoset or thermoplastic polymer, the ionic polymer interpenetrating at least the first hydrophobic thermoset or thermoplastic polymer. In some embodiments, the water-swellable IPN or semi-IPN has properties mimicking stiffness and lubricity properties of natural cartilage and may be adapted and configured to replace cartilage in a joint. In alternative embodiments, materials incorporating other forms of interpenetrating polymer network or semi-interpenetrating polymer network are employed in the making of said prosthetic menisci.

Where magnetic resonance imaging is employed in the diagnosis of meniscal injury or deterioration or in the measurement of the size and shape of joint components, typical imaging units employ knee coils of the several types manufactured by Fonar Corporation of 110 Marcus Drive, Melville, N.Y. 11747, USA are employed. Similar units are manufactured by many other large manufacturers of imaging equipment. Images of knee cartilages are alternatively generated using high frequency ultrasound, frequencies of 200 MHz giving a maximum resolution approaching 10 microns. Images of knee cartilages are also alternatively generated using diffraction-enhanced X-ray imaging.

Any of the methods described herein for enhancement of the lubricity of said working surfaces is also optionally employed to minimise the possibility of implant infection as a result of the implantation of said prosthetic menisci. In a first embodiment, said menisci are made with an outer layer containing a long-acting, broad-spectrum bacteriostatic antibiotic such as minocycline. In another embodiment, said antibiotics contained in the outer layers of said prosthetic menisci are diterpenes, either synthetic or derived from botanical sources. In another embodiment, said antibiotics contained in the outer layers of said prosthetic menisci are cationic peptide antibiotics. In another embodiment, said antibiotics contained in said outer layers of said prosthetic menisci are combined with a class of compounds known as 2-aminoimidazoles, which have been demonstrated as effective in the dispersion of biofilms. In another embodiment, silver nanoparticles are incorporated into the surfaces of said prosthetic menisci or into polymer coatings applied to said surfaces. In another embodiment, said silver nanoparticles incorporated into the surfaces of said prosthetic menisci are made by reducing silver nitrate in solutions of tea extract or epicatechin, the cytotoxicity of said silver nanoparticles being thereby substantially reduced. In another embodiment, iron oxide nanoparticles are incorporated into the surfaces of said prosthetic menisci or into polymer coatings applied to said surfaces. In another embodiment, synthetic metallomolecules7 [Fe 2 L 3] 4+(L=C 25H 20N 4) are incorporated into the surfaces of said prosthetic menisci or into polymer coatings applied to said surfaces. Said molecules take the form of tetracationic supramolec-ular cylinders, the molecular shaping and high positive charge of which enhance their ability to bind with DNA, thereby interrupting cell replication and exerting a powerful bacteriostatic effect.

Any feasible combination of any part of the apparatus and/or any part of the method described herein should be taken to be disclosed by the specification.

### REFERENCES

1. Fu F. H., Thompson W. O. Motion of the Meniscus During Knee Flexion. In: Mow V. C., Arnoczky S. P., Jackson D. W., eds. Knee Meniscus: Basic and Clinical Foundations. New York: Raven Press, 1992: Chapter 6, FIGS. 20 and 21.
2. Vedi V., Williams A., Tennant S. J., Spouse E., Hunt D. M., Gedroyc W. M. W. (1998). Meniscal Movement: An In-vivo Study Using Dynamic MRI. The Journal of Bone & Joint Surgery (Br) 1999; 81-B37-41.
3. Hamamoto K., Tobimatsu M. D., Zabinska-Uroda K. (2004). Magnetic Resonance Imaging Evaluation of the Movement and Morphological Changes of the Menisci During Deep Knee Flexion. J. Phys. Ther. Sci. Vol. 16 No. 2, 2004
4. Williams III P. F., Powell G. L., Love B., Ishihara A., Nakabayashi N., Johnson R., LaBerge W. (1994). Fabrication and Characterization of Dipalmitoylphosphatidylcholine-attracting Elastomeric Material for Joint Replacements.
5. Keeley et al. International Patent. No. WO 2008/140703 A2. Synthetic Peptide Materials for Joint Reconstruction, Repair and Cushioning.
6. Pasqui D., Atrei A., Barbucci R. (2007). A Novel Strategy to Obtain a Hyaluronan Mionoloayer on Solid Substrates. Biomacro-molecules, 2007, 8 (11), pp 3531-3539
7. Moldrheim E., Hannon M. J., Meistermann I., Rodger A., Sletten E. Interaction Between a DNA Oligonucleotide and a Dinuclear Iron (II) Supramolecular Cylinder; and NMR and Molecular Dynamics Study. Journal of Biological Inorganic Chemistry, Volume 7, Numbers 7-8, 770-780, DOI: 10.1007/s00775-002-0354-2

1-73. (canceled)

74. A method of making prosthetic knee menisci to be implanted in place of deteriorated or damaged native menisci to prevent injury to the articular cartilage of the femoral and tibial condyles and, thereby, to prevent the progressive development of osteoarthritis; said prosthetic menisci being sequentially injection moulded in suitable dies from a suitable biocompatible elastomeric polymer material having physical characteristics similar to those of the native menisci and comprising femoral condyle contact panel, tibial condyle contact panel, upper panel and outer panel; sheets of thin, flexible polymer film reinforcement material being embedded medially in all said panels, the edges of each said sheet (excepting at the inner edges of said femoral and tibial condyle contact panels) being fixed or joined to adjacent sheets prior to moulding of said panels; during moulding of said menisci, the outer surfaces of said sheets of reinforcement material of said panels being supported in the desired shaping by a suitable female die and the inner parts of said panels moulded between said sheets of reinforcement material and a suitable male die which defines the interior shapings of said panels; said male die being built up from multiple parts to enclose internal reinforcements in the form of a plurality of sheets of thin, flexible polymer film reinforcement material to be maintained free and un-embedded, the outer edges of said internal reinforcements being joined to said reinforcement material of said outer panel and the inner edges being left free; said male die then being retained in place and the outer parts of said panels moulded between said sheets of reinforcement material and a replacement larger female die which defines the exterior shapings of said panels; said male die and said larger female die being retained in place and a third die used to define the exterior shapings of optional extensions of said panels; said male die being made such as to orientate the moulded forms of said femoral condyle and tibial condyle contact panels parallel to each other and to expose the inner surfaces of said sheets of reinforcement material of said contact panels in zones towards their free, inner edges; said moulded panels being removed from said dies, said internal reinforcements lightly tensioned to maintain them in flat, sheet form, the inner, free edges of said internal reinforcements being joined and said joined parts being captured between and joined to said exposed zones of said reinforcement material of said femoral and tibial condyle contact panels as the inner edges of said contact panels are brought together, all said components being permanently joined by fusing or through the use of a suitable bonding agent, thereby transforming said prosthetic menisci into closed, hollow forms; said hollow forms being sized for femoral and tibial condylar surfaces and filled or inflated after implantation by injection into them of a gel or settable polymer to shape them into congruence with the femoral and tibial condyles; having internal reinforcement for strength and durability; the bearing surfaces of said menisci being treated chemically and/or physically to improve the efficiency of lubrication by synovial fluid and to enhance the wear characteristics of said bearing surfaces; and said menisci being restricted in translation within the interarticular space by anchorage of their anterior and posterior horns to the tibia and by the provision of secondary locating elements.

75. The method of claim 74 in which the inner edges of said inner parts of said femoral and tibial condyle contact panels adjacent said exposed zones of said inner surfaces of said sheets of reinforcement material are given complementary shapings such that, when said inner edges of said contact panels are brought together, said complementary shapings register accurately with each other; optionally in which said prosthetic menisci are manufactured in a range of sizes, small increments between successive members of a range permitting accurate sizing to the condyles of a prospective recipient.

76. The method of claim 74 in which reinforced radial extensions are provided on the outer edges of said tibial condyle contact panels to provide a means of attaching said menisci; optionally in which said reinforced radial extensions are provided only in discrete locations; optionally in which said reinforced radial extensions are made elastic in a form similar to that of said horns of said prosthetic menisci; optionally in which the reinforcements of said radial extensions are radial extensions of said sheets of reinforcement material of said tibial condyle contact panels; optionally in which said inner parts and said outer parts of said femoral and tibial condyle contact panels are made with a thickness in the range 0.5 to 5.0 millimetres, said thickness being greater in some parts and lesser in others.

77. The method of claim 74 in which said sheets of reinforcement material are made from a thin, flexible, polymer film, including Kevlar®, with a thickness in the range 0.01 to 1.0 millimetres; optionally in which said sheets of reinforcement material are provided with a plurality of apertures to facilitate bonding, fusing, moulding or encapsulation to, in or by said biocompatible elastomeric polymer material, said apertures being of a shape and arrangement to leave intact zones capable of satisfactorily carrying the radial and circumferential loads applied to said reinforcement material; optionally in which said sheets of reinforcement material are made from a woven material comprising warp and weft of monofilaments, or which are optionally fused, or spun or braided, multi-filament yarn made from materials having a suitable tensile strength; optionally in which said reinforcements are made from Kevlar® with a diameter in the range 0.01 to 1.0 millimetres, the woven form of said reinforcements facilitating their shaping to suit the various parts of said prosthetic menisci; optionally in which said woven reinforcements are made with a warp arranged generally radially and weft following the curved shaping of said prosthetic menisci, gaps approximately trapezoidal in shape being left between said warp and weft, said gaps having a maximum distance between opposing vertices in the range 0.5 to 5 millimetres; optionally in which, following their weaving and prior to encapsulation, said woven reinforcements are coated with a thin layer of polymer material to fix the arrangement; optionally in which the inner edges of said femoral and tibial condyle contact panels are extended such that, when said extended edges are joined together, they create either an annular zone or an apron covering all or part of the interiors of said prosthetic menisci.

78. The method of claim 74 in which said menisci are made with anterior and posterior horns and locating straps made from a suitable biocompatible elastomer, said horns and locating straps being employed to anchor said menisci and strengthened by one or more layers of fully encapsulated reinforcements; optionally in which said horns, webs, locating straps or aprons optionally comprise one of more layers of thin, flexible film reinforcement material of high tensile strength encapsulated in a biocompatible, elastomeric polymer material, said reinforcement material having a thickness in the range 0.05 to 0.5 millimetres and made with a plurality of parallel, sinusoidal cuts aligned generally with the designed load path, the application of force to said load path causing said sinusoidal parts to straighten, as limited by the simultaneous elastic compliance of said encapsulating polymer material; optionally in which said elastic extension is limited to the range zero to 20 percent; optionally in which said elastic extension is limited to the range 10 to 50 percent; optionally in which said reinforcement material is Kevlar®; optionally in which said prosthetic knee menisci are made with one or more layers of reinforcement material passing fully along their arcuate lengths and along the lengths of said horns and encapsulated in a suitable biocompatible elastomeric polymer; and with a web optionally connecting said horns and reinforced by one or more layers of said reinforcement material made contiguous with that of said meniscus proper; and with closely-spaced, discrete, radially-arranged strips of said reinforcement material distributed throughout the arcuate length of said menisci and preferably interleaved with said layers of reinforcement material, said strips preferably being fixed at their inner ends to a common band of said reinforcement material embedded in the inner edge of said meniscus and at their outer edges to the upper edge of said outer panel reinforcement material; optionally in which said radially-arranged strips of reinforcement material are optionally extended inwardly to form the basis of an apron covering the internal area of a said meniscus, said apron being made continuous with said web; optionally in which said reinforcement material optionally takes the form of a thin, flexible film material or a woven material, both of suitable tensile strength, said reinforcement material acting to accommodate stresses generated by normal functional loadings and which tend to stretch and enlarge a said meniscus and urge its extrusion from a joint.

79. The method of claim 78 in which reinforcements of said horns and said locating straps in the form of monofilaments or spun or braided, multi-filament yarns pass into and form a strong connection with said menisci by being woven back and forth through suitable apertures provided in the end parts of said film reinforcement material to provide a strong connection; optionally in which the encapsulation material of said horns is optionally the same base material from which said prosthetic menisci are made.

80. The method of claim 74 in which said female die for shaping the external surfaces of said femoral and tibial condyle contact panels are finely finished so as to provide a glass-smooth finish to said surfaces; optionally in which the external surfaces of either or both said femoral and tibial condyle contact panels are provided with thin layers of a macroporous, softer and more compliant polymer material, said layers preferably have a thickness in the range 0.1 to 2.0 millimetres and pores having a width in the range 50 to 300 nm, said surface being better able to achieve microelastohydrodynamic lubrication; optionally in which said prosthetic menisci made from a softer, more compliant base material are employed temporarily during restoration of femoral or tibial articular cartilage and are subsequently replaced with menisci made from a harder base material.

81. The method of claim 78 in which reinforcement material of said horns is made in a sinusoidal arrangement by being secured in that form to a work surface with a plurality of pins and coated with an initial coating of an elastomeric polymer material to fix the arrangement, said pins being removed after setting of said initial coating and said coated reinforcement assembly joined to attachment means at the ends of said horns and to said reinforcements of said prosthetic menisci, following which said horns are fully encapsulated with said elastomeric polymer material in a die or mould; optionally in which, when loaded, said sinusoidally-arranged reinforcements of said horns tend to straighten, as permitted by the elastic compliance of said encapsulating elastomeric polymer material, thereby providing a capacity to accommodate shock loadings.

82. The method of claim 74 in which webs optionally joining the horns of said prosthetic menisci are reinforced by film or woven sheet material made from a strong, cross-linked polymer material with a thickness in the range 0.05 to 0.5 millimetres, the material of said reinforcements preferably being Kevlar® and providing a maximum elastic extension in the range zero to 20 percent.

83. The method of claim 74 in which bone anchors are installed in the anterior and posterior intercondylar areas of the tibia, said installed bone anchors preferably being inclined towards the appropriate horns of said prosthetic menisci at an angle of between 30 degrees and 60 degrees to the axis of the tibia; optionally in which said bone anchors take the form of cortical orthopaedic screws with parallel sides on which is formed helically-arranged fluting; optionally in which said bone anchors take the form of cancellous bone anchors, with tapered body parts and deeper fluting than said cortical orthopaedic screws; optionally in which said bone anchors are preferably made to be self-tapping and are made from titanium treated to promote osseointegration; optionally in which said bone anchors are each made with a hollow bore to receive the distal end of an attachment fitting, said attachment fitting being secured to a said bone anchor by the screwing of a threaded distal parts into a thread in said bore or by complementary, bayonet-type connection means; optionally in which said bayonet-type connection means comprise lugs on the distal ends of said attachment fittings, said lugs passing down opposed, axial grooves provided in said bore to engage short, circumferentially-arranged grooves, the end parts of said circumferentially-arranged grooves being deflected towards the open end of said bore and acting to capture said lugs against the urging of a spring applied to said attachment fittings via a captive piston slideably accommodated in said bore and permitted a restricted range of movement by the inner ends of one of more radially-arranged pins fixed in the walls of said bone anchor and extending into said bore to engage an annular recess formed in said piston; optionally in which suitable shaped apertures are provided in the ends of said bone anchors, said apertures being engaged by a suitable tool to screw said bone anchor into said tibia; optionally in which suitable apertures, hexagonal parts or flatted parts are provided at the proximal ends of said attachment fittings to be engaged by a suitable tool for the purpose of engaging said attachment fitting with said bone anchor; optionally in which said bone anchors are inserted into said tibia with sufficient depth such that the proximal ends of said attachment fittings in their installed positions are more or less level with the surface of said tibia; optionally in which the proximal ends of said attachment fittings are connected to the horns of said menisci by means of connection pieces permanently attached to the ends of said horns by said reinforcements of said horns passing through a curved attachment slot formed in the proximal end of each said connection piece, the shaping of said attachment slot ensuring that tensional forces applied by said reinforcements will be normal to each point of contact; optionally in which said attachment fitting is provided with a circumferential groove just below its head, the distal end of said connection piece being formed into a pair of folded-back or returned, parallel claws which engage said circumferential groove, a sharply folded-back, springy tab formed between said claws elastically engaging the rounded top of the head of said attachment fitting to secure said claws in place on said attachment fitting, cuts of suitable length being made in said connection piece to increase the springiness of said springy tab; optionally in which said connection piece is made with a cylindrical part which is rotationally accommodated in a bore formed in the proximal part of said attachment fitting, said cylindrical part being captured by the inner ends of one or more pins fixed in the walls of said attachment fitting engaging an annular groove formed in said cylindrical part, said cylindrical part preferably being joined to an expanded proximal end part incorporating a curved attachment slot by a narrow rod; optionally in which said connection pieces are permitted limited angular displacement across the surface of said tibial plateaus; optionally in which, where revision of said prosthetic meniscus is required, said attachment fitting is readily detached from said bone anchor by unscrewing it from said threaded bore of said bone anchor or releasing said bayonet-type connection, said bone anchor being left permanently in place in said tibia; optionally in which a prosthetic meniscus is secured to said tibia by the ends of its horns being permanently attached to the proximal ends of said connection pieces and the distal ends of said connection pieces being removably attached to said bone anchors screwably inserted into said tibia; optionally in which said bone anchors are made solid with a rounded cap and circumferential groove at their proximal ends, said rounded cap and circumferential groove being engaged by said connection piece; optionally in which said bone anchors are made substantially solid and with a coaxial bore having an internal circumferential groove, said connection pieces having at their distal ends a cylindrical part incorporating a ball lock-type locking means, said locking means incorporating two or more hardened balls urged into said bore circumferential groove to lock said cylindrical part into place in said bore by the outward displacement between them by a sprung rod; inward displacement of said rod against the urging of said spring force bringing a circumferential groove formed in said rod into coincidence with said balls, permitting them to withdraw from said bore circumferential groove and, thereby, permitting said cylindrical part to be withdrawn from said bore; optionally in which the distal end of said connection piece extends into a bore in said cylindrical part and serves as said ball displacement rod, tension applied to said connection piece applying additional force to radially displace said balls and, thereby, to lock said cylindrical part more strongly into said bore in said bone anchor, a compression force applied to said connection piece permitting said cylindrical part to be withdrawn from said bore, a suitable spring preferably being provided at the inner end of said bore to displace said cylindrical part from said bore when said balls are withdrawn.

84. The method of claim 74 in which, during an implantation procedure, said prosthetic menisci are filled or inflated with a biocompatible, viscous liquid or a liquids settable to a gel or to a rubbery solid, suitable viscous liquids preferably have a dynamic viscosity in the range 5 to 50 Pa·s at normal body temperature with higher viscosity liquids being employed in thinner-walled forms of said prosthetic menisci, examples of biocompatible, viscous liquids being diglycerols, polyglycerols and silicones; optionally in which said biocompatible gels include the chemically gellable, aqueous, biocompatible, polysaccharide gel composition taught by Bardonnet et al in WO/2002/57355 or other biocompatible, self-gelling, natural or synthetic polymers, such as rapid transition polymers which can be made liquid at lower temperatures while forming a gel at normal body temperatures, reversibility of the gelling process by cooling facilitating drainage and collapse of a prosthetic meniscus in the case where revision is required; optionally in which said biocompatible, settable polymers include the bioactive and biocompatible polyurethane-butanediol-glycosaminoglycan copolymer taught by Masters et al in WO 2008/134468 A1, the hardness of such materials in their set state being readily manipulated by manufacturers; optionally in which said filling or inflation medium is preferably injected into a said prosthetic meniscus by means of a suitable syringe via a single site in said outer panel or, if required, via multiple injection sites in said outer panel situated above, below and between said un-embedded, internal reinforcements, said medium flowing through a plurality of apertures in said internal reinforcements, gelling or setting of said filling or inflation medium acting to capture said internal reinforcements in a gel or solid matrix, thereby opposing any tendency for a said meniscus to be extruded from a joint; optionally in which, because of the nature of said filling or inflation medium there is danger of its leakage, a suitable thin, self-sealing membrane is provided on the inner surface of said outer panel, the location of the safe injection sites so created being marked or otherwise indicated on the exterior surface of said outer panel, the self-sealing nature of said membrane acting to positively close the aperture created by insertion of said syringe upon its withdrawal from an injection site.

85. The method of claim 74 in which, where the lubricity of the external surfaces of said femoral and tibial condyle contact panels is to be enhanced by the generation thereon of a layer of hyaline cartilage, a layer of suitable, highly porous scaffold material is prepared and fused or otherwise fixed to or formed on said surfaces and seeded with articular chondrocytes immediately before implantation of said prosthetic menisci, said hyaline cartilage being allowed to generate in-situ.

86. The method of claim 74 in which, to provide improved hydrophilicity and lubrication of the external surfaces of said femoral and tibial condyle contact panels, hyaluronic acid is introduced into said elastomeric polymer material from which said prosthetic meniscus is made or is applied to its working surfaces using methods adapted from those taught by Kim et al in U.S. Pat. No. 4,500,676, in a first method, hyaluronic acid being dissolved in an appropriate solvent and the obtained solution mixed with a polymer solution or an emulsion, an article being formed from the mixture or the mixture being applied as a coating; suitable solvents for dissolving hyaluronic acid being water, dimethylsulfoxide and dimethylformamide, the surface of the polymeric substrate being able to be activated, as can the hyaluronic acid; in a second method, hyaluronic acid being added in a solid form, preferably as a powder, to a polymer solution, an article being formed from the mixture or the mixture is applied as a coating; in a third method, a particulate material being coated with hyaluronic acid from solution and the coated particles being introduced into a polymer solution, an article being formed from the mixture or the mixture applied as a coating, examples of suitable particulate materials being ion exchange resins, silica, alumina, or the like; in a fourth method, in any of the three preceding methods described, the hyaluronic acid is cross-linked before or after mixing with said polymer material, using various cross-linking agents, a typical cross-linking agent being the polymer CX100.

87. The method of claim 74 in which, to provide improved hydrophilicity and lubrication of the external surfaces of said femoral and tibial condyle contact panels, an ultrathin layer of polysaccharide is generated on said surfaces by silanizing said surfaces and applying to them a derivatized polysaccharide created by conjugating a silane alkylic chain with hyaluronan, said surfaces preferably being first modified by corona discharge followed by application of an organo-functional silane or, alternatively, UV treatment in the presence of alkylsilanes is used to introduce Si—H groups onto said surfaces.

88. The method of claim 74 in which, to provide improved hydrophilicity and lubrication of the external surfaces of said femoral and tibial condyle contact panels, in a method adapted from those taught by Swan et al in U.S. Pat. No. 5,414,075, Swan in U.S. Pat. No. 6,278,018 and Chappa et al in U.S. Pat. No. 7,736,689, a grafting reagent is used to form a polymeric layer on a support surface, and particularly on a porous support surface, in a manner that provides and/or preserves desired properties (such as porosity) of the surface, the reagent and method able to be used to provide a thin, conformable, uniform, un-crosslinked coating having desired properties onto the surface of a preformed, and particularly a porous, polymeric substrate, the method including the steps of, (a) providing a porous support surface, (b) providing a nonpolymeric grafting reagent comprising a photoinitator group, (c) providing one or more polymerizable monomers adapted to be contacted with the surface in the presence of the grafting reagent and to be polymerized upon activation of the photoinitiator and, (d) applying the grafting reagent and monomer(s) to the surface in a manner and under conditions, suitable to coat the surface with said grafting reagent and to cause the polymerization of monomers to the surface upon activation of the grafting reagent; optionally in which the following laboratory procedure is adapted for the surface modification of polyurethane (PU) by application of acrylamide/acrylamidomethylpropane sulfonic acid (AMPS) with the restrained, multifunctional reagent for surface modification (RMRSM) as taught by Swan et al: RMRSM being prepared according to the method described in Example 1 as taught by Swan et al, a coating solution being prepared by dissolving an amount of RMRSM at 1 g/l in 100% isopropyl alcohol (IPA), polyurethane rods 5 cm long being wiped with an 99% purity IPA-soaked, lint-free cloth and allowed to dry, the cleaned PU rods being dipped into said RMRSM solution, removed at a steady rate (approximately 2 cm/sec) and allowed to dry for at least 5 minutes, said rods, after application of RMRSM to them, being placed in a solution containing a mixture of monomers (acrylamide 3% or 7% and AMPS 7% or 3% respectively, weight to volume) in deionized (DI) water, approximately 8 ml of said monomer mixture being placed in a glass syringe containing a stopcock in the bottom to prevent the solution from draining out, said PU rods being placed in said syringe and said monomer solution deoxygenated by sparging nitrogen gas through it for a minimum of 10 minutes, the deoxygenated solution containing said PU rods being exposed to UV light (150 seconds in the 320-500 nm wavelength range, intensity of light measured by radiometer as being approximately 20 mw/cm2 measured at a distance of 2.5-3.0 cm from the light source), following exposure to said UV light, said samples being removed from said monomer solution and washed thoroughly to remove any residual unbound monomer, the described method producing a durable, lubricious, biocompatible coating, preserving surface porosity and ensuring the maintenance of natural lubrication functionality; optionally in which the following laboratory procedure is adapted for the surface modification of polyurethane (PU) by application of acrylamide/acrylamidomethylpropane sulfonic acid (AMPS) with the surface coating agent (SCA) as taught by Swan: a reagent of the structure shown as Compound II being prepared in the manner taught by Swan, a coating solution being prepared containing 5 mg/ml of Compound II in DI water, 5 cm polyurethane rods being cleaned with 99% purity IPA using a lint-free cloth and allowed to dry, the cleaned PU rods being placed in a clear glass tube containing the Compound II solution, said rods being incubated in the solution at room temperature for approximately five minutes, following said incubation, the substrate in said Compound II solution being illuminated with a doped mercury vapor lamp (three minutes at an intensity of 1-1.5 mW/cm2 in the wavelength range of 330-340 nm) to activate the photoreactive groups present in Compound II, thereby attaching it to the surfaces of said rods as a base coat, following said UV curing, said rods being rinsed in DI water for approximately 30 seconds prior to graft polymerization, in which the rods coated with the Compound II base coat are placed in 8.0 ml of a mixture of acrylamide and AMPS monomer salt solution contained in a 10 ml glass syringe, said monomer mixture and substrate being deoxygenated by sparging nitrogen gas through said syringe for 10 minutes, said sparging being continued while said solution is irradiated with UV light (150 seconds at an intensity of 10 mW/cm2 in the 330-340 nm wavelength, measured by radiometer at a distance of 3.0 cm from the end of the light guide), following said UV irradiation, said rods being removed from said grafting solution and washed in DI water to remove any unbound monomer, the described method producing a durable, lubricious, biocompatible coating, preserving surface porosity and ensuring the maintenance of natural lubrication functionality.

89. The method of claim 74 in which the external surfaces of said femoral and tibial contact panels are functionalized by the conjugation onto them of bioactive agents using an atmospheric glow plasma discharge method taught by Roy and Raja in WO 2007/008755 A2, said method able to be employed to functionalize the surfaces of polymer materials, including biopolymers and biodegradable polymers including PMMA, poly(lactide-co-glycolide) (PLGA), polylactic acid (PLA), polyglycolic acid (PGA), polycaprolactone (PCL), or polyethylene glycol (PEG) having two- or three-dimensional surfaces, by the attachment to said surfaces of bioactive agents in the form of charged and radical species as said surfaces are passed through said APG plasma discharge, for the purposes of the present invention, said functionalized surfaces being loaded with hyaluronic acid, DPPC or the like; optionally in which, where the material from which said prosthetic menisci are made is not fully a crystalline polymer, the lubricity of the external surfaces of said femoral and tibial condyle contact panels is enhanced by impregnation of the said surfaces with hyaluronic acid or other lubricating component of synovial fluid, said impregnation taking place after fabrication and being effected by dispersing said lubricating component in finely divided dry form over the meniscal surface to be treated and subjecting said surface to supercritical carbon dioxide at a temperature above 30.1° C. and pressure in the range 150 to 180 bar, said lubricating component being dissolved by said super-critical carbon dioxide and diffused into said surface, satisfactory impregnation normally being achieved in less than three minutes; optionally in which the lubricity of the external surfaces of said femoral and tibial condyle contact panels is enhanced by providing means to trap a layer of synovial fluid on said surfaces, said trapping effect being created by the generation on said surfaces of a dense layer of electrospun fibres, said electrospun fibres being spun from molten polymer precursors and entrained in a flow of heated gas at a temperature in the range 0 to 25 percent higher than the glass transition temperature of the spun fibres, said fibres impacting a polymer surface being treated and fusing at the point of contact, the greater part of the lengths of said fibres remaining unattached to said treated surface; optionally in which the lubricity of the external surfaces of said femoral and tibial condyle contact panels is enhanced by the generation on said surfaces of polymer brushes, said polymer brushes being grown using surface initiated polymerisation (SIP) or atom-transfer radical polymerisation (ATRP) with the initiator molecule transferred to the substrate using microcontact printing, alternate methods of synthesising polymer brushes also being employed; optionally in which the lubricity of the external surfaces of said femoral and tibial condyle contact panels is enhanced by making the material of said panels with appropriate surface modifying end groups incorporated into the material from which said prosthetic menisci are made and rendering said surfaces hydrophilic and attractive to the lubricating constituents of synovial fluid.

90. The method of claim 74 in which the surfaces of said femoral and tibial condyle contact panels are enhanced by a method adapted from that taught by Ward in EP 2 213 293 A2 for the immobilization of biologically-active entities, including proteins, peptides, and polysaccharides at a surface of a polymer body, a variety of simple hydrophobic and hydrophilic endgroups enabling the achievement of useful changes in surface properties of polymers including biostability, protein adsorption, abrasion resistance, bacterial adhesion and proliferation, fibroblast adhesion and coefficient of friction; polymers of a number of types being able to be used as base polymers for carrying the covalently bonded self-assembling monolayer (SAM) endgroups of the present invention, a “self-assembling moiety”-containing polymer molecule endgroup being defined as an endgroup that spontaneously rearranges its positioning in a polymer body to position the moiety on the surface of the body, which positioning effects a reduction in interfacial energy, the endgroup structure able to comprise one or more chemical groups, chains, or oligomers that spontaneously assemble in the outermost monolayer of the surface of the polymer body, or to comprise one or more chemical groups, chains, or oligomers that spontaneously assemble within the bulk of the polymer body, the polymer bulk being defined as the region within the polymer body that is at least one monolayer away from the outermost monolayer of the polymer body surface, the method providing for the configuration of the nanostructure, supramolecular structure, and/or conformation of a molecular monolayer at a surface of a polymer body at an interface; optionally in which the method involves contacting a polymer body surface with a separate medium to form an interface under conditions that facilitate the delivery of endgroup molecular moieties to the polymer body surface and maximize the resulting concentration of head groups in the outermost surface, said delivery being, in part, due to the interaction of chemical groups, chains, or oligomers in the endgroup moieties, said endgroup molecular moieties being covalently or ionically bonded to a polymer in the body and including one or more chemical groups, chains, or oligomers that spontaneously assemble in the outermost monolayer of the surface of said polymer body or one or more chemical groups, chains, or oligomers that spontaneously assemble within that portion of said polymer body that is at least one monolayer away from the outermost monolayer of said polymer body surface; in accordance with the method, the endgroups being bonded to said polymers through a divalent oligomeric chain, have at least 5 repeat units capable of self-assembly with corresponding chains on adjacent molecules of the polymeric composition, suitable structures for the spacer chains being found in the SAM and silane literature, in general, self-assembling spacer chains suitable for polymer endgroups for performance of the method being those that self assemble when present in self-assembling thiol or silane SAMs, the surface-modifying endgroup moieties being delivered to the polymer body surface by their spontaneous diffusion to the surface region of the polymer body or by their rearrangement or repacking in the surface layer of the polymer body, the polymer comprising the surface-modifying endgroup moieties in the polymer body making up the entirety, or a major portion, of the body and having a weight average molecular weight in the range 5000-5,000,000 daltons, preferably in the range 50,000-1,000,000 daltons, optional delivery of surface-modifying endgroups to said polymer body surface being accomplished by adding a surface-modifying additive (SMA) to the polymer just described, with said additive comprising a second polymer that is covalently or ionically bonded to said surface-modifying endgroup moieties; when delivery of the surface-modifying endgroup moiety to the polymer surface is accomplished by adding an SMA to the polymer to be modified, the useful molecular weight range of the polymer used as an SMA may be lower: 1000-5,000,000 daltons and preferably in the range 5000 to 200,000 daltons, as the SMA is typically used in low bulk concentrations, e.g. less than 15 weight-%, and preferably about 1 to 5 weight-%, so that the physical-mechanical properties of the base polymer/SMA blend will be largely determined by the base polymer being modified, a very low SMA molecular weight possibly causing the SMA to be fugitive from the polymer being modified, e.g. by leaching or even volatilizing from the surface of the base polymer in use, particularly when exposed to fluids, vacuum, and/or high temperatures, candidate SMA polymers with molecular weight less than 5000 being generally unsuitable and must be tested for their permanence in the base polymer before use in applications; alternatively, delivery of surface-modifying endgroup moieties to the polymer body surface or other substrate to be modified is able to be accomplished by coating, plasma treatment, painting, or otherwise topically treating the surface of a pre-formed body with a material comprising a second polymer covalently or ionically bonded to the surface-modifying endgroup moieties of the present invention, other well known methods also being employed in treating said working surfaces of said prosthetic menisci.

91. The method of claim 74 in which the lubricity of the external surfaces of said femoral and tibial condyle contact panels is enhanced by application of a method taught by Smith in U.S. Pat. No. 6,221,108 in which the polyurethane material of said surfaces are treated with Ringer's solution in a heated bath for at least 96 hours at a temperature of between 30° C. and 37° C.; optionally in which the lubricity of the external surfaces of said femoral and tibial condyle contact panels is enhanced by making said surfaces from a semi-interpenetrating or interpenetrating network of at least two materials, one material to provide the characteristic of toughness and one to provide the characteristic of lubricity, the method employed being that taught by Myung et al in US Patent Application No. 20100010114, said materials including compositions of a water-swellable interpenetrating polymer network (IPN) or semi-IPN of a hydrophobic thermoset or thermoplastic polymer and an ionic polymer, the compositions exhibiting a lower coefficient of friction than the hydrophobic thermoset or thermoplastic polymer, said IPN or semi-IPN being more water-swellable, exhibiting higher resistance to creep, and/or exhibiting a higher conductivity and permeability than said hydrophobic thermoset or thermoplastic polymer, in some embodiments, said IPN or semi-IPN being formed by diffusing an ionizable monomer precursor solution into the hydrophobic thermoset or thermoplastic polymer and polymerizing the monomers to form the ionic polymer, in some embodiments, the composition also including water, which may form a hydration gradient from a first portion of the composition to a second portion of the composition, an electrolyte may be dissolved in the water; said IPN or semi-IPN may also be negatively charged; said hydrophobic thermoset or thermoplastic polymer is optionally physically entangled or chemically crosslinked with the ionic polymer; in some embodiments, said hydrophobic thermoset or thermoplastic polymer has ordered and disordered domains, and the ionic polymer may be disposed in the disordered domains; in some embodiments said hydrophobic thermoset or thermoplastic polymer may be selected from the group consisting of polyurethane, polymethyl methacrylate, polydimethylsiloxane, and acrylonitrile butadiene styrene, said ionic polymer able to be, for example, a poly(acrylic acid) or poly(sulfopropyl methacrylate) or combinations or derivatives thereof; said ionic polymer may include carboxylate groups and/or sulfonate groups, said ionic polymer preferably being hyaluronic acid; in some embodiments, said ionic polymer forms a concentration gradient from a first portion of the composition to a second portion of the composition, the concentration gradient optionally providing a stiffness and/or hydration gradient within the composition; some embodiments including a second hydrophobic thermoset or thermoplastic polymer which is optionally disposed in a layer separate from the first hydrophobic thermoset or thermoplastic polymer or may be diffused throughout the first hydrophobic thermoset or thermoplastic polymer; another embodiment provides a process for producing a water-swellable IPN or semi-IPN from an hydrophobic thermoset or thermoplastic polymer including the following steps of: placing an ionizable monomer solution in contact with a solid form of the hydrophobic thermoset or thermoplastic polymer, diffusing the ionizable monomer solution into the thermoset or thermoplastic polymer; and polymerizing the ionizable monomers to form a ionic polymer inside the thermoset or thermoplastic polymer, thereby forming the IPN or semi-IPN; some embodiments including the step of swelling the IPN or semi-IPN with water, for example, to form a hydration gradient from a first portion of the composition to a second portion of the composition, the method possibly including the step of swelling the IPN or semi-IPN with an electrolyte solution; various embodiments of the method including the steps of chemically crosslinking or physically entangling said hydrophobic thermoset or thermoplastic polymer with said ionic polymer; in embodiments in which said hydrophobic thermoset or thermoplastic polymer has ordered and disordered domains, the method may include the step of swelling the disordered domains with the ionizable monomer solution prior to the polymerizing step; in some embodiments, said hydrophobic thermoset or thermoplastic polymer is selected from the group consisting of polyurethane, polymethyl methacrylate, polydimethylsiloxane, and acrylonitrile butadiene styrene, said ionizable monomer solution may be an acrylic acid solution and may comprise monomers with carboxylate groups and/or sulfonate groups; in some embodiments, the method includes the step of forming a concentration gradient of said ionic polymer within the IPN or semi-IPN through regioselective diffusion of the ionizable monomer solution through said hydrophobic thermoset or thermoplastic polymer to, for example, provide a stiffness and/or hydration gradient within the composition; some embodiments of the method possibly including, prior to the polymerizing step, the steps of placing said ionizable monomer solution in contact with a solid form of a second hydrophobic thermoset or thermoplastic polymer and diffusing the ionizable monomer solution into the second hydrophobic thermoset or thermoplastic polymer, in such embodiments, the second hydrophobic thermoset or thermoplastic polymer may be in a separate layer adjacent to said first hydrophobic thermoset or thermoplastic polymer or may be diffused within the first hydrophobic thermoset or thermoplastic polymer; some embodiments include the step of changing the IPN or semi-IPN from a first shape to a second shape, such as by heating the IPN or semi-IPN; some embodiments have a second hydrophobic thermoset or thermoplastic polymer adjacent to said first hydrophobic thermoset or thermoplastic polymer, the ionic polymer interpenetrating at least the first hydrophobic thermoset or thermoplastic polymer; in some embodiments, the water-swellable IPN or semi-IPN has properties mimicking stiffness and lubricity properties of natural cartilage and may be adapted and configured to replace cartilage in a joint; in alternative embodiments, materials incorporating other forms of interpenetrating polymer network or semi-interpenetrating polymer network are employed in the making of said prosthetic menisci.

92. The method of claim 74 in which, where magnetic resonance imaging is employed in the diagnosis of meniscal injury or deterioration or in the measurement of the size and shape of joint components, typical imaging units employ knee coils of the several types manufactured by Fonar Corporation of 110 Marcus Drive, Melville, N.Y. 11747, USA and similar units made by many other manufacturers of imaging equipment; optionally in which images of knee cartilages are generated using high frequency ultrasound, frequencies of 200 MHz giving a maximum resolution approaching 10 microns; optionally in which images of knee cartilages are generated using diffraction-enhanced X-ray imaging.

93. The method of claim 74 in which any of the described methods for enhancing the characteristics of the external surfaces of said prosthetic menisci are also optionally employed to minimise the possibility of implant infection as a result of the implantation of said prosthetic menisci; said prosthetic menisci being made with an outer layer containing a long-acting, broad-spectrum bacteriostatic antibiotic such as minocycline, diterpenes (either synthetic or derived from botanical sources) or cationic peptide antibiotics, said antibiotics optionally being combined with the class of compounds known as 2-aminoimidazoles, which have been demonstrated as effective in the dispersion of biofilms; in another embodiment, silver nanoparticles are incorporated into the surfaces of said prosthetic menisci or into polymer coatings applied to said surfaces; in another embodiment, silver nanoparticles incorporated into the surfaces of said prosthetic menisci are made by reducing silver nitrate in solutions of tea extract or epicatechin, the cytotoxicity of said silver nanoparticles being thereby substantially reduced; in another embodiment, iron oxide nanoparticles are incorporated into the surfaces of said prosthetic menisci or into polymer coatings applied to said surfaces; in another embodiment, synthetic metallomolecules [Fe 2 L 3] 4+(L=C 25H 20N 4) are incorporated into the surfaces of said prosthetic menisci or into polymer coatings applied to said surfaces, said molecules taking the form of tetracationic supramolecular cylinders, the molecular shaping and high positive charge of which enhance their ability to bind with DNA, thereby interrupting cell replication and exerting a powerful bacteriostatic effect.
